SEQUENCE LISTING (13 sequences)

SEQ ID NO: 1 - DNA, 2732 bp, Homo sapiens
aacaacatcctgggattgggacccactttctgggcactgctggccagtcccaaaatggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaattggacgggagacagagtgacgcactgactcacctagaggctgggacgtgggtagggatttagcatgctggaaataactggcagtaatcaaacgaagacactgtccccagctaccagctacgccaaacctcggcattttttgtgttattttctgactgctggattctgtagtaaggtgacatagctatgacatttgttaaaaataaactctgtacttaactttgatttgagtaaattttggtttt

SEQ ID NO: 2 - DNA, 1455 bp, Homo sapiens
gtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtg

SEQ ID NO: 3 - DNA, 783 bp, Homo sapiens
gtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatcc

SEQ ID NO: 4 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 5 - protein, 260 aa, Homo sapiens
  VARIANT 211: Xaa = Asn or Glu
VYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPSSGPTYQCLKGTGENYRGNVATVTVSGHTCQHWSAQTPHTHXRTPENFPCKNLDENYCRNPDGKRAPWCHTTNSQVRWEYCKIPSCDSSPV

SEQ ID NO: 6 - protein, 333 aa, Homo sapiens
MDHKEVILLFLLLLKPGQGDSLDGYISTQGASLFSLTKKQLAAGGVSDCLAKCEGETDFVCRSFQYHSKEQQCVIMAENSKTSSIIRMRDVILFEKRVYLSECKTGIGNGYRGTMSRTKSGVACQDWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAP

SEQ ID NO: 7 - protein, 484 aa, Homo sapiens
VYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRV

SEQ ID NO: 8 - DNA, 57 bp, Homo sapiens
  misc_binding (1)...(57): angio forward
cgtacctagggaattcaagcttactagtgccgccgccatggatgcaatgaagagagg

SEQ ID NO: 9 - DNA, 39 bp, Homo sapiens
  misc_binding (1)...(39): angio reverse
gtcagtcgactctagaagtggatctttaaggtggtgctg

SEQ ID NO: 10 - DNA, 549 bp, Homo sapiens
cacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaag

SEQ ID NO: 11 - protein, 183 aa, Homo sapiens
HSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 12 - DNA, 96 bp, Homo sapiens
atggatgcaatgaagagagggctctgctgtgtgctgctgctgtgtggagcagtcttcgtttcgcccagcggtaccggatccttatttgaaaagaaa

SEQ ID NO: 13 - protein, 32 aa, Homo sapiens
MDAMKRGLCCVLLLCGAVFVSPSGTGSLFEKK

(19) United States
(12) Patent Application Publication
(10) Pub. No.: US 2004/0071659 A1
(43) Pub. Date: Apr. 15, 2004

(54) Viral-mediated delivery and in vivo expression of polynucleotides encoding anti-angiogenic proteins

(76) Inventors: Betty Chang, Cupertino, CA (US); Wei Wei Wu, Redwood City, CA (US); James McArthur, San Carlos, CA (US); Salil D. Patel, South Plainfield, NJ (US); Karin Jooss, San Francisco, CA (US); Michael J. Mendez, Albuquerque, NM (US); Brian A. Donahue, Los Altos, CA (US)

Correspondence Address: Steven B. Kelber, Esq., Piper Rudnick LLP, 1200 19th Street, NW, Washington, DC, 20036, US

(21) Appl. No.: 10/135,872

(22) Filed: Apr. 29, 2002

Related U.S. Application Data
(60) Provisional application No. 60/287,673, filed on Apr. 30, 2001.
(60) Provisional application No. 60/370,634, filed on Apr. 5, 2002.

(51) Int. Cl. A61K 48/00; C12N 7/00; C12N 15/861

(52) U.S. Cl. 424/93.2; 435/456; 435/235.1

(57) ABSTRACT

The present invention provides anti-angiogenic compositions and methods, based on gene delivery vectors that include the coding sequence for an anti-angiogenic compound.

### FIELD OF THE INVENTION

The present invention relates to viral-mediated delivery and in vivo expression of polynucleotides encoding anti-angiogenic proteins, in particular to the use of recombinant adeno-associated viral vectors and adenoviral vectors used to deliver genes encoding angiostatin and endostatin.

### BACKGROUND

It is generally accepted that tumor development requires the secretion by cancer cells of soluble mediators, so-called “tumor angiogenic factors”, which activate the formation of new blood vessels. The discovery of vascular endothelial growth factor (VEGF) and of new proteases had led to the identification of the key actors of tumor angiogenesis. The elucidation of their mechanisms of action has allowed the design of new therapeutic strategies, including the use of anti-angiogenic compounds.

Several potent physiological angiostatic factors have been described that are proteolytic fragments of larger native proteins. Proteins that fall into that category include Angiostatin, endostatin, platelet factor-4 and the 16-kD fragment of prolactin. Angiostatin and endostatin are known to have strong anti-angiogenic activity. It has been theorized that if these compounds could be introduced in the right amount at the right place and time, they could be used to inhibit vascularization of a growing tumor, effectively starving the tumor of nutrients, thereby restricting its growth and, perhaps, preventing subsequent pathology. Previously-tried therapies include delivery of anti-angiogenic agents directly to the tumor, direct bolus injection and sustained systemic delivery. Sustained systemic delivery from an external pump is considered to be significantly more effective than bolus injection. But sustained delivery faces a number of practical difficulties including the risk of infection associated with catherization. In addition, direct delivery of protein may not be feasible if the protein is labile. Overall, experimental treatments involving the administration of anti-angiogenic agents directly to the tumor site showed limited efficacy.

Adeno-associated virus (AAV) is a helper-dependent human parvovirus which is able to infect cells latently by chromosomal integration. Various studies from 1970 to 1986 demonstrated that 15-30% of immortalized cells could be infected latently with wildtype AAV, and that the AAV genome was chromosomally linked. Moreover, a similar ability for integration was demonstrated for recombinant AAV in immortalized tissue culture cells (Hermonat and Muzyczka, Proc. Natl. Acad. Sci. (1984) 81:6466-70; and Tratschin et al., Mol. Cell. Biol. (1985) 5:3251-60). In 1988, recombinant AAV transduction of primary hematopoietic stem cells was achieved (LaFace et al., Virology (1988) 60:483-86). Recently, the preferred site of wild-type AAV integration was demonstrated to be in a region of human chromosome 19 (see, e.g., Kotin et al., Proc. Natl. Acad. Sci. U.S.A. (1991) 87:2211-15; Kotin et al., EMBO. 1. (199) 11:5071-78; and Samulski et al., EMBO J. (1991) 10:3941-50). Because of its ability to integrate chromosomally and its nonpathogenic nature, AAV has significant potential as a human gene therapy vector.

Adenoviruses are a relatively well characterized, homogeneous group of viruses. Roughly 100 different adenoviruses, including nearly 50 serotypes isolated from humans, have been identified to date. Most common serotypes of adenovirus vectors are nonpathogenic, physically and genetically stable, can be grown to very high titers (concentrated stocks with 1011 to 1012 PFU/ml of infectious virus are easy to obtain) and easily purified by isopycnic centrifugation in CsCl gradients. The adenovirus genome is readily manipulated by recombinant DNA techniques, and the proteins encoded by foreign DNA inserts that are expressed in mammalian cells will usually be appropriately glycosylated or phosphorylated, unlike recombinant proteins expressed in bacteria, yeast, and some insect cells. Although human adenovirus vectors replicate most efficiently in human cells of epithelial origin, these viruses infect almost any mammalian cell and express at least some viral genes. Unlike retroviruses, adenovirus vectors will infect, and are expressed in, nonreplicating cells. Thus, adenoviral-based vectors may be useful for gene delivery, expression, and gene therapy. Recombinant adenovirus vectors have been used to deliver angiostatin-like molecules to glioma tumor cells in combination with radiotherapy with some success (Griscelli, F., et al., Proc. Natl. Acad. Sci (2000) 97:6698-6703; and Griscelli, F. et al., Proc. Natl. Acad. Sci (1998) 95:6367-6372).

Recent reports have indicated that sustained delivery of anti-angiogenic compounds may improve their therapeutic activity in tumor models, as compared to cyclical delivery of these same agents.

Despite advances in cancer treatment strategies, lack of efficacy and/or significant side effects due to the toxicity of currently used chemotherapeutic agents remains a problem. Drug toxicity can be severe enough to result in life-threatening situations, which require administration of drugs to counteract side effects, and may result in the reduction and/or discontinuation of the chemotherapeutic agent, which can impact negatively on the patient's treatment and/or quality of life. Gene therapy strategies have been attempted and are the subject of ongoing clinical trials, but have not yet proven to have clinical usefulness. Accordingly, there remains a need for improved cancer treatment regimens which address the deficiencies in current therapeutic approaches. The present invention addresses this need. More specifically, there is currently a need for vectors and methods that allow virally-mediated delivery and expression of anti-angiogenic factors in a subject. Preferably, such methods exhibit efficient transduction of target cells, good therapeutic yield of anti-angiogenic factor, low liver toxicity and result in a reduction in endothelial cell proliferation and a reduction in the proliferation and migration of tube forming cells (i.e., anti-angiogenesis).

### SUMMARY OF THE INVENTION

The present disclosure provides vectors and methods that allow virally-mediated delivery and expression of anti-angiogenic factors in a subject wherein such methods result in efficient viral transduction of target cells and a good therapeutic yield of the anti-angiogenic factors.

In one aspect, the invention provides recombinant viral vectors for obtaining angiostatin activity, wherein the vectors comprise a promoter capable of expressing human angiostatin operably linked to a structural gene encoding one or more domains of human angiostatin.

In a related aspect, the invention provides recombinant adenovirus (rAV) or adeno-associated virus (rAAV) vectors for expressing an anti-angiogenic compound, wherein the vector comprises a promoter capable of expressing a biologically active form of the anti-angiogenic compound operably linked to a structural gene encoding the anti-angiogenic compound.

In one preferred embodiment, the anti-angiogenic compound is angiostatin.

In another preferred embodiment, the anti-angiogenic compound is endostatin

In a further preferred embodiment, the promoter is the EF-1 alpha promoter.

The invention is further directed to methods for inhibiting angiogenesis in a mammalian subject, by administering a replication-defective viral vector comprising a DNA sequence encoding angiostatin operably linked to a promoter, in a manner effective to result in expression of a biologically active form of angiostatin.

In a related aspect, the invention is directed to methods for inhibiting angiogenesis in a mammalian subject, by administering a recombinant adeno-associated virus (rAAV) vector comprising a DNA sequence encoding an anti-angiogenic compound operably linked to a promoter, wherein a biologically active form of the anti-angiogenic compound is expressed.

In one preferred embodiment, the anti-angiogenic compound is angiostatin.

In another preferred embodiment, the anti-angiogenic compound is endostatin

In a further preferred embodiment, the promoter is the EF-1 alpha promoter.

In one aspect of the invention, the vector is administered in vivo, into the portal vasculature of the mammal or by the intravenous route.

These and other objects and features of the invention will be more fully appreciated when the following detailed description of the invention is read in conjunction with the accompanying drawings.

### BRIEF DESCRIPTION OF THE DRAWINGS

FIGS. 1A and B are diagrams of exemplary recombinant adenoviral (rAV) vectors, in particular, E1 deleted and E1/E4 deleted AV vectors) encoding human angiostatin and endostatin polypeptides (FIG. 1A) and kringle domains 1-3 of human angiostatin under control of a CMV or EF-1alpha promoter (FIG. 1B).

FIG. 2 is an image of a Western blot illustrating the results of expression analysis of ΔE1-AV-angiostatin and ΔE1/ΔE4-AV-angiostatin, in vitro and in vivo. Lanes 2,10 were loaded with 2 μg of angiostatin from Pichia pastoris; lanes 3, 9 are samples from ΔE1-AV-angiostatin transduced cells or mice serum; lanes 4,8 are samples from ΔE1-AV-GFP transduced cells; lanes 5, 7 are samples from ΔE1/ΔE4-AV-angiostatin transduced cells; and lane 6 is a sample from ΔE1/ΔE4-AV-GFP injected mice serum.

FIG. 3 shows the inhibition of human umbilical vein endothelial cells (HUVEC) cell migration by ΔE1 and ΔE1/ΔE4 adenovirus produced angiostatin. Conditioned media from ΔE1-AV or ΔE1/ΔE4-AV encoding either lacZ or angiostatin were incubated with HUVEC cells and assayed for its effect on the migration of HUVEC cell by a modified Boyden chamber migration assay. Conditioned media was diluted to identical concentrations with cell culture media. HUVEC cells were applied onto the upper chamber along with the cell culture media. The lower chamber contained 10 ng/ml of VEGF as a chemo attractant. Migrated cells were stained, scored and averaged as described in Methods and Materials.

FIG. 4 illustrates the anti-tumor efficacy of ΔE1-AV-angiostatin and ΔE1/ΔE4-AV-angiostatin in the B16B1/6 tumor model. Two doses (5×10¹¹ vp (H) or 5×10¹⁰ vp (L)) of ΔE1-AV or ΔE1/ΔE4-AV encoding either GFP or angiostatin, as well as angiostatin protein at 100 μg per mouse/day as a bolus intravenous injection for 10 consecutive days in C57B1/6 mice two days after injection of 5×10⁴ of B16B1/6 tumor cells. Two weeks following the tumor cell injections, mice were sacrificed and lungs harvested for metastasis assessment. Both ΔE1-AV-angiostatin, ΔE1/ΔE4-AV-angiostatin treatment reduced surface lung metastasis (n=6, p<0.005 in Student t-test). Similar animal experiments were repeated at least 3 times.

FIG. 5 illustrates angiostatin expression in the serum of mice at various time points (7-99 days) following intravenous injection of ΔE1-AV-angiostatin or ΔE1/ΔE4-AV-at 5×10¹⁰ vp into mice.

FIG. 6 is a graphic depiction of the survival of mice in the B16B1/6 tumor model from 0 to 34 days following intravenous injection of 5×1010 vp of ΔE1/ΔE4-AV with either angiostatin or LacZ under control of the CMV promoter into mice.

FIGS. 7A and B are diagrams of rAAV vectors encoding human angiostatin and endostatin polypeptides under control of the EF1-α and LSP promoters, respectively.

FIG. 8 is an image of a Western blot illustrating in vitro expression of angiostatin following transduction of 293 cells with AAV EF1α-angiostatin or AAV EF1α-GFP (AAV-EF1α-angio sup; AAV-EF1α-GFP sup.) and in vivo expression of angiostatin in mouse serum (AAV-EF1α-angio serum; AAV-EF1α-GFP serum; AAV-LSP-angio serum; AAV-LSP-GFP serum), respectively.

FIG. 9 is a graphic illustration of serum angiostatin levels in mice at week 1-week 5, following injection of AAV EF1α-angiostatin into Balb/c nu/nu mice at doses of 5×10¹⁰ vp (5e10 vp), 2×10¹¹ vp (2e11 vp), and 3×10¹¹ vp (3e11 vp), (n=10).

FIG. 10A is a schematic illustration of the B16F10-luc metastasis model, where tumors are evaluated twice weekly by imaging with a Xenogen Imaging System and lung metastases are counted on day 21 (D21).

FIG. 10B depicts images of Balb/c nu/nu mice in a B16F10-luc metastasis model, which were injected intravenously with 2×10⁵ B16F10 tumor cells on day 0 and 5×10¹⁰ particles of rAAV encoding either GFP (AAV-GFP) or angiostatin (AAV-Angio) on day −21 via the portal vein. A Xenogen Imaging System was used twice per week to evaluate tumor burden based on in vivo bioluminescence in the mice.

FIG. 10C is a graphic depiction of the number of lung metastases on day 21 (D21) in the B16F10 metastasis model following day 0 portal vein injection of PBS or 5×10¹⁰ particles of rAAV encoding either GFP (AAV-EF1α-GFP) or angiostatin (AAV-EF1α-angio).

FIG. 11A is a graphic depiction of the survival of mice in a B16F10 metastasis model from day 0 to day 70 following day 0 portal vein injection of 5×10¹⁰ particles of rAAV encoding either AAV-EF1α-GFP (gfp) or AAV-EF1α-angio (angio).

FIG. 11B is a graphic depiction of the concentration of serum angiostatin (ng/ml) in Balb/c nu/nu mice from day 0 over time for 8 weeks following day 0 injection of 5×10¹⁰ particles of rAAV encoding either AAV-EF1α-GFP (gfp) or AAV-EF1α-angio (angio).

FIG. 12A is a schematic illustration of the Lewis Lung Carcinoma Resection Tumor Model where tumors are evaluated twice weekly by imaging with a Xenogen Imaging System and lung metastases are counted on 28 days (D28) after primary tumor removal.

FIG. 12B is a graphic depiction of CCD photon counts obtained using a Xenogen Imaging System. The backs of mice were injected with 10⁶ Lewis Lung Carcinoma Cells (LLC) and one week later all mice that had developed primary tumors were injected with 5×10¹⁰ AAV-GFP (GFP) or AAV-angiostatin (angiostatin) vp. CCD photon counts were evaluated 2 weeks after primary tumor resection (n=10).

FIG. 15A is a schematic illustration of the Pancreatic Islet Carcinoma Model in RIP-Tag mice (as further described below).

FIG. 15B is a graphic depiction of the average pancreatic islet tumor burden (mm3) following sacrifice at week 13 after portal vein injection of 7-week-old RIP-Tag mice with AAV-angiostatin (angio) or AAV-GFP (gfp) at 1.5×10¹¹ vp (n=6).

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al., Molecular Cloning: A Laboratory Manual (Second Edition), Cold Spring Harbor Press, Plainview, N.Y., 1989 and Ausubel F M et al., Current Protocols in Molecular Biology; John Wiley & Sons, New York, N.Y. 1993, for definitions and terms of the art. It is to be understood that this invention is not limited to the particular methodology, protocols, and reagents described, as these may vary.

All publications cited herein are expressly incorporated herein by reference for the purpose of describing and disclosing compositions and methodologies which might be used in connection with the invention.

The term “angiogenesis”, as used herein refers to the sprouting of blood vessels from pre-existing blood vessels, characterized by endothelial cell proliferation and the proliferation and migration of tube forming cells. Angiogenesis can be triggered by certain pathological conditions, such as the growth of solid tumors and metastasis.

The terms “anti-angiogenic compound”, “anti-angiogenic factor”, “anti-angiogenic polypeptide” and “anti-angiogenic protein”, as used herein refer to a compound or factor that inhibits angiogenesis. i.e., the sprouting of blood vessels from pre-existing blood vessels, characterized by endothelial cell proliferation and the proliferation and migration of tube forming cells. It follows that anti-angiostatic activity means inhibition of angiogenesis.

The term “biologically active form”, as used herein relative to an anti-angiogenic compound or factor, such as human angiostatin or endostatin means any form of the anti-angiogenic compound or factor that exhibits anti-angiogenic activity. Anti-angiogenic activity may be evaluated using any of a number of assays routinely employed by those of skill in the art, including, but not limited to, an endothelial cell migration assay, a Matrigel tube formation assay, endothelial and tumor cell proliferation assays, apoptosis assays and aortic ring assays, as further described below.

The term “exposing”, as used herein means bringing an anti-angiogenic factor-encoding vector in contact with a target cell. Such “exposing”, may take place in vitro, ex vivo or in vivo.

The term “vector”, as used herein, refer to a nucleic acid construct designed for transfer between different host cells. An “expression vector” or “gene therapy vector” refers to a vector that has the ability to incorporate and express heterologous DNA fragments in a foreign cell. A cloning or expression vector may comprise additional elements, for example, the expression vector may have two replication systems, thus allowing it to be maintained in two organisms, for example in human cells for expression and in a prokaryotic host for cloning and amplification. The term vector may also be used to describe a recombinant virus, e.g., a virus modified to contain the coding sequence for an anti-angiogenic compound or factor.

The term “replication defective” as used herein relative to a viral gene therapy vector of the invention means the viral vector cannot further replicate and package its genomes. For example, when the cell of a subject are infected with rAAV virions, the heterologous gene is expressed in the patient's cells, however, due to the fact that the patient's cells lack AAV rep and cap genes and the adenovirus accessory function genes, the rAAV is replication defective and wild-type AAV cannot be formed in the patient's cells.

The term “operably linked” as used herein relative to a recombinant DNA construct or vector means nucleotide components of the recombinant DNA construct or vector that are directly linked to one another for operative control of a selected coding sequence. Generally, “operably linked” DNA sequences are contiguous, and, in the case of a secretory leader, contiguous and in reading frame. However, enhancers do not have to be contiguous.

As used herein, the term “gene” or “coding sequence” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “sequence identity” means nucleic acid or amino acid sequence identity in two or more aligned sequences, aligned using a sequence alignment program. The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90 or 95% or more sequence identity to a PKR sequence, as described herein.

Exemplary computer programs that can be used to determine identity between two sequences include, but are not limited to, the suite of BLAST programs, e.g., BLASTN, BLASTX, TBLASTX, BLASTP and TBLASTN, all of which are publicly available on the Internet. See, also, Altschul, S. F. et al., 1990 and Altschul, S. F. et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. [See, Altschul, et al., 1997.]

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm−5° C. (5° below the Tm of the probe); “high stringency” at about 5-10° below the Tm; “intermediate stringency” at about 10-200 below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993, expressly incorporated by reference herein). An example of high stringency conditions includes hybridization at about 42° C. in 50% formamide, 5×SSC, 5× Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

The terms “transcriptional regulatory protein”, “transcriptional regulatory factor” and “transcription factor” are used interchangeably herein, and refer to a cytoplasmic or nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to the DNA response element.

As used herein, the terms “stably transformed”, “stably transfected” and “transgenic” refer to cells that have a non-native (heterologous) nucleic acid sequence integrated into the genome. Stable transformation is demonstrated by the establishment of cell lines or clones comprised of a population of daughter cells containing the transfecting DNA. In some cases, “transformation” is not stable, i.e., it is transient. In the case of transient transformation, the exogenous or heterologous DNA is expressed, however, the introduced sequence is not integrated into the genome.

As used herein, the terms “biological activity” and “biologically active”, refer to the activity attributed to a particular protein in a cell line in culture. It will be appreciated that the “biological activity” of such a protein may vary somewhat dependent upon culture conditions and is generally reported as a range of activity. Accordingly, a “biologically inactive” form of a protein refers to a form of the protein that has been modified in a manner that interferes with the activity of the protein as it is found in nature.

The terms “apoptotic cell death” and “apoptosis”, as used herein refer to any cell death that results from, or is related to, the complex cascade of cellular events that occur at specific stages of cellular differentiation and in response to specific stimuli. Apoptotic cell death is characterized by condensation of the cytoplasm and chromatin condensation in the nucleus of dying cells. The process is associated with fragmentation of DNA into multiples of 200 base pairs and degradation of RNA as well as proteolysis in an organized manner without sudden lysis of the cell as in necrotic cell death.

As used herein, the terms “tumor” and “cancer” refer to a cell that exhibits a loss of growth control and forms unusually large clones of cells. Tumor or cancer cells generally have lost contact inhibition and may be invasive and/or have the ability to metastasize.

The term “administering”, as used herein refers to delivering a gene therapy vector encoding an anti-angiogenic compound or factor to the cells of a subject. Such administering may take place in vivo, in vitro or ex vivo.

As used herein, “effective amount” relative to a vector encoding an anti-angiogenic compound or factor refers to the vector administered to a mammalian subject, either as a single dose or as part of a series of doses and which is effective to result in an improved therapeutic outcome of the subject under treatment.

As used herein “treatment” of an individual or a cell is any type of intervention used in an attempt to alter the natural course of the individual or cell. Treatment includes, but is not limited to, administration of e.g., a vector encoding an anti-angiogenic compound or factor, a pharmaceutical composition, alone or in combination with other treatment modalities generally known in the art. The “treatment” may be performed prophylactically, or subsequent to the initiation of a pathologic event.

As used herein, the term “improved therapeutic outcome” relative to a cancer patient refers to a slowing or diminution of the growth of cancer cells or a solid tumor, or a reduction in the total number of cancer cells or total tumor burden.

II. Anti-Angiogenic Compounds

The invention is directed to gene therapy compositions and methods for use in inhibiting angiogenesis. Preferred anti-angiogenic compounds include endostatin, angiostatin, platelet factor-4, the 16-kD fragment of prolactin, sFlt-1 (a soluble fragment of fms-like tyrosine kinase 1 receptor), sKDR (soluble fragment of kinase insert domain receptor), sVEGFR3 (soluble extracellular forms of VEGFRs), TRAIL (tumor necrosis factor—related apoptosis-inducing ligand), thrombospondin, interferon alpha and (PEDF) pigment epithelium-derived factor, as well as factors involved in signaling pathways that lead to expression/production of these compounds. The gene therapy compositions and methods of the present invention provide vectors comprising anti-angiogenic genes, the expression products of which slow inhibit or prevent angiogenesis.

“Angiostatin” refers to a 38 kilodalton N-terminal fragment of plasminogen that inhibits neovascularization, as described in detail in O'Reilly et al., Cell (1994) 79:315-328, the contents of which are incorporated herein by reference. The 2732 bp mRNA sequence for human plasminogen is found at GenBank Accession No. X05199 (presented herein as SEQ ID NO:1). Angiostatin was first isolated from mice bearing a Lewis lung carcinoma, and was identified as a 38 kD proteolytic fragment of plasminogen that encompasses the four kringles of the parent molecule. The amino acid sequence for K1-5 of human angiostatin corresponds to amino acids 98 to 581 of the human plasminogen polypeptide sequence (presented herein as SEQ ID NO:7). Individual recombinant kringle domains 1-3 (K1-3; SEQ ID NO:5) of angiostatin were all shown to possess inhibitory activity against endothelial cell proliferation, whereas kringle 4 did not. Angiostatin K1-4 was also found to be no more potent as an inhibitor of endothelial cell proliferation than angiostatin K1-3. Angiostatin delivery to tumor cells correlated with an induction of apoptosis in the endothelial cells in vitro and also in vivo. Subcutaneous bolus injections of Angiostatin had proven to be efficacious in six different tumor models in mice without apparent toxicity. However, recent animal studies have shown that continuous low dose administration of anti-angiogenic proteins demonstrate much better efficacy than bolus injections of the recombinant proteins. The continuous delivery of endostatin via an Alzet osmotic pump showed sustained systemic concentrations of the protein leading to increased tumor regression, and a close to 10 decrease in the dose required achieve the same anti-tumor efficacy as the single daily bolus administration of angiostatin (Kisker, et al., 2001 Cancer Res., 61(20):7669-74). Continuous administration of angiostatin inhibited corneal angiogenesis and the growth of both primary and metastatic tumors in a dose dependent manner. Similarly, continuous administration of Angiostatin was significantly better than bolus injection at inhibiting the growth of both primary and metastatic tumors (Drixler et al., 2000, Cancer Res. 60(6):1761-5). On the other hand, protein injections suffer from a potential loss of activity due to stability issues dependent upon the protein administered. These problems can potentially be overcome by viral gene therapy.

Angiostatin includes human as well as other mammalian versions of the polypeptide. In particular, human angiostatin commonly refers to amino acids 98 to 581 of the 810 amino acid sequence of human plasminogen (which is presented herein as SEQ ID NO:4). See, e.g., Sim, B. K et al., 1997, Cancer Res. 57 (7), 1329-1334; Mulichak, A. M. et al., 1991 Biochemistry 30 (43), 10576-10588 and GenBank Accession No. P00747.

Truncated forms of angiostatin that retain its anti-angiogenic properties are also included in the definition of angiostatin (See, e.g., SEQ ID NO:5 and SEQ ID NO:6). A preferred form of angiostatin is the polypeptide sequence comprising amino acids 98 through 357 of human plasminogen (SEQ ID NO:5). It is also preferred that angiostatin include a signal sequence, an example of which is presented herein as SEQ ID NO:13 (encoded by the nucleotide sequence presented herein as SEQ ID NO:12).

The present invention provides gene therapy vectors that include the coding sequence for a biologically active form of angiostatin. In one preferred embodiment the angiostatin is in the form of K1-3. In other embodiments the coding sequence for a biologically active form of angiostatin is provided as K1-5, K1-4 or K5.

“Endostatin” refers to a 20 kilodalton C-terminal fragment of collagen XVIII that inhibits angiogenesis. Endostatin is described in detail in O'Reilly et al., Cell, 1997, 88:277-85, the contents of which are incorporated herein by reference. Endostatin, as used herein, includes human as well as other mammalian versions (e.g., mouse) of the polypeptide. Truncated forms of endostatin that retain its anti-angiogenic properties are also included in the definition. A particularly preferred form of endostatin is the 183-amino acid polypeptide sequence, presented herein as SEQ ID NO:1.

The present invention also provides gene therapy vectors that include the coding sequence for a biologically active form of endostatin. In a preferred embodiment the full-length 20 kd endostatin coding sequence is included in the vector.

Vascular endothelial growth factor (VEGF) is produced when tumors grow to a certain size (2-3 mm) and become hypoxic, or oxygen starved. VEGF participates in a signaling process that triggers the activation, division, and migration of endothelial cells that line blood vessel walls, resulting in the growth of capillaries from the blood vessels and into the VEGF-secreting tumor. (See, e.g., Ferrara, N., 1999, Curr. Top. Microbiol. Immunol. 237, 1-30; Risau, W., 1997, Nature 386, 671-674; and Yancopoulus, G. D. et al., 2000, Nature 407, 242-248.) Accordingly, inhibitors and soluble receptors for VEGF are included within te3h definition of anti-angiogenic compounds.

Examples of naturally occurring and recombinant forms of the exemplary anti-angiogenic compounds, angiostatin and endostatin can be found in the patent and scientific literature including, U.S. Pat. Nos. 6,306,819, 6,218,517, 6,024,688, 5,945,403, 5,837,682, 5,801,012, 5,776,704 and 5,733,876. However, the present invention is not limited to any particular anti-angiogenic compound coding sequence. Accordingly, any known, or later discovered DNA sequence coding for a biologically active anti-angiogenic compound can be included in the vectors of the present invention.

The invention further contemplates variant and partial sequence forms of anti-angiogenic compounds, so long as the variant or partial sequence form maintains anti-angiogenic activity.

Sequence variants include nucleic acid molecules that encode the same polypeptide as is encoded by the anti-angiogenic compounds described herein. Thus, where the coding frame of the anti-angiogenic gene is known, it will be appreciated that as a result of the degeneracy of the genetic code, a number of coding sequences can be produced. For example, the triplet CGT encodes the amino acid arginine. Arginine is alternatively encoded by CGA, CGC, CGG, AGA, and AGG. Therefore it is appreciated that such substitutions in the coding region fall within the sequence variants that are covered by the present invention.

It is further appreciated that such sequence variants may or may not hybridize to the parent sequence under conditions of high stringency. This would be possible, for example, when the sequence variant includes a different codon for each of the amino acids encoded by the parent nucleotide. Such variants are, nonetheless, specifically contemplated and encompassed by the present invention.

In accordance with the present invention, also encompassed are sequences that are at least 80%, preferably 85%, more preferably 90%, 95% or 98% identical to the angiostatin and endostatin coding sequences described herein.

III. Gene Delivery Vectors

The present invention contemplates the use of any of a variety of viral vectors for introduction of a gene encoding an anti-angiogenic proteins into mammalian cells. Viruses can efficiently transfect their own DNA into a host cell, resulting in production of new viral particles. In constructing viral vectors, non-essential genes tare replaced with a gene encoding an anti-angiogenic proteins. Such vectors typically include retroviruses (including lentiviruses), adenoviruses, adeno-associated viruses and herpes simplex virus type 1.

Adenovirus gene therapy vectors are known to exhibit strong transient expression, excellent titer, and the ability to transduce dividing and non-dividing cells in vivo (Hitt M M and Graham F L, 2000 Adv in Virus Res 55:479-505). The recombinant AV vectors of the instant invention comprise: (1) a packaging site enabling the vector to be incorporated into replication-defective AV virions; and (2) an anti-angiogenic compound coding sequence. Other elements necessary or helpful for incorporation into infectious virions, such as the 5′ and 3′ AV ITRs, the E2 and E3 genes, etc., may also be included.

Replication-defective AV virions encapsulating the recombinant AV vectors of the instant invention are made by standard techniques known in the art using AV packaging cells and packaging technology. Examples of these methods may be found, for example, in U.S. Pat. No. 5,872,005. An anti-angiogenic compound-encoding gene is commonly inserted into adenovirus in the deleted E1A and E1B region of the virus genome. Preferred adenoviral vectors for use in practicing the invention will not express one or more wild-type AV polypeptides. Preferably, the replication-defective AV virions will not express one or more of the following AV gene products: E1a, E1b, E2, E3, E4. Particularly preferred embodiments are virions that are E1-deleted, E1/E4 deleted or E1/E3/E4 deleted. Such vectors are typically used together with packaging cell lines that complement the functions of E1, E2A, E4 and optionally the E3 gene regions. See, e.g. U.S. Pat. Nos. 0.5,872,005, 5,994,106, 6,133,028 and 6,127,175, expressly incorporated by reference herein.

The recombinant AAV vectors of the instant invention comprise: (1) a packaging site enabling the vector to be incorporated into replication-defective AAV virions; and (2) an anti-angiogenic compound coding sequence. Other elements necessary for incorporation into infectious virions, such as the 5′ and 3′ AAV ITR (inverted terminal repeats) may also be included.

AAV vectors for use in practicing the invention are constructed such that they include, as operatively linked components in the direction of transcription, control sequences including transcriptional initiation and termination sequences, and the coding sequence for an anti-antigenic compound or biologically active fragment thereof. These components are bounded one the 5′ and 3′ end by functional AAV ITR sequences. By “functional AAV ITR sequences” is meant that the ITR sequences function as intended for the rescue, replication and packaging of the AAV virion. Hence, AAV ITRs for use in the vectors of the invention need not have a wild-type nucleotide sequence (e.g., as described in Kotin, Hum. Gene Ther., 5:793-801, 1994), and may be altered by the insertion, deletion or substitution of nucleotides or the AAV ITRs may be derived from any of several AAV serotypes.

The gene therapy vectors of the invention typically include heterologous control sequences, which include, but are not limited to, constitutive promoters, such as the cytomegalovirus (CMV) immediate early promoter, the RSV LTR, the MoMLV LTR, and the PGK promoter; tissue or cell type specific promoters including mTTR, TK, HBV, hAAT, regulatable promotes, enhancers, etc. Preferred promoters include the LSP promoter (Ill, C R et al., 1997, Blood Coagul. Fibrinolysis 8S2:23-30), the EF1-alpha promoter (Kim D W et al. 1990. Gene. 91(2):217-23 and Guo Z S et al. 1996. Gene Ther. 3(9):802-10),

Recombinant AAV (rAAV) vectors and virions for use in practicing the present invention may be produced using standard methodology, known to those of skill in the art. An AAV vector is a vector derived from an adeno-associated virus serotype, including without limitation, AAV-1, AAV-2, AAV-3, AAV-4, AAV-5, AAV-6, AAV-7, etc. Preferred AAV vectors have the wild type REP and CAP genes deleted in whole or part, but retain functional flanking ITR sequences. A recombinant AAV is a virus that has been genetically altered, e.g., by the addition or insertion of a heterologous nucleic acid sequence into the particle.

Typically, an AAV expression vector is introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large rep proteins (Rep78 and Rep68), typically by mutating the start codon following p5 from ATG to ACG. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500; 6,040,183; and 6,093,570, expressly incorporated by reference herein.

In practicing the invention, host cells for producing rAAV virions include mammalian cells, insect cells, microorganisms and yeast. In one preferred embodiment, the human embryonic kidney cell line, 293 are used in the practice of the present invention. 293 cells have been transformed with adenovirus type-5 DNA fragments and expresses the adenoviral E1A and E1B.

Host cells can also be packaging cells in which the AAV rep and cap genes are stably maintained in the host cell. Host cells can be producer cells in which the AAV vector genome is stably maintained in the packaging cell. Packaging and producer cells may be derived from 293, A549 or HeLa cells.

AAV vectors are purified by standard techniques including CsCl gradient ultracentrifugation, iodixinol gradient centrifugation, heparin affinity chromatography, ion-exchange chromatography, and/or size exclusion chromatography. Purified AAV vectors can be formulated in a number of formulations including phosphate buffered saline, Hank's balanced salt solution, Ringer's solution, sucrose, and mannitol. The present invention contemplates the inclusion of a gene regulation system for the controlled expression of an anti-angiogenic compound or factor. Gene regulation systems are useful in the modulated expression of a particular gene or genes. In general, gene regulation systems or switches include a chimeric transcription factor that has a ligand binding domain, a transcriptional activation domain and a DNA binding domain. The domains may be obtained from virtually any source and may be combined in any of a number of ways to obtain a novel protein. A regulatable gene system also includes a DNA response element which interacts with the chimeric transcription factor. This element is located adjacent the gene to be regulated.

Exemplary gene regulation systems that may be employed in practicing the present invention include, the Drosophila ecdysone system (Yao and Evans, 1996, Proc. Nat. Acad. Sci., 93:3346), the Bombyx ecdysone system (Suhr et al., 1998, Proc. Nat. Acad. Sci., 95:7999), the Valentis GeneSwitch® synthetic progesterone receptor system which employs RU-486 as the inducer (Osterwalder et al., 2001, Proc Natl Acad Sci 98(22):12596-601); the Tet™ & RevTet™ Systems (B D Biosciences Clontech), which employs small molecules, such as tetracycline (Tc) or analogues, e.g. doxycycline, to regulate (turn on or off) transcription of the target (Knott A et al., Biotechniques 2002, 32(4):796, 798, 800); ARIAD Regulation Technology which is based on the use of a small molecule to bring together two intracellular molecules, each of which is linked to either a transcriptional activator or a DNA binding protein. When these components come together, transcription of the gene of interest is activated. Ariad has two major systems: a system based on homodimerization and a system based on heterodimerization (Rivera et al., 1996, Nature Med. 2(9):1028-1032; Ye et al., 2000, Science 283: 88-91)

Preferred gene regulation systems for use in practicing the present invention are the ARIAD Regulation Technology and the Tet™ & RevTet™ Systems.

The gene therapy vectors and constructs described above may be introduced into cells using standard methodology known in the art. Such techniques include transfection using calcium phosphate, micro-injection into cultured cells (Capecchi, Cell 22:479-488 [1980]), electroporation (Shigekawa et al., BioTechn., 6:742-751 [1988]), liposome-mediated gene transfer (Mannino et al., BioTechn., 6:682-690 [1988]), lipid-mediated transduction (Felgner et al., Proc. Natl. Acad. Sci. USA 84:7413-7417 [1987]), and nucleic acid delivery using high-velocity microprojectiles (Klein et al., Nature 327:70-73 [1987]).

IV. In Vitro Evaluation Of Angiogenesis

The effectiveness of a given vector encoding an anti-angiogenic compound or factor may be evaluated in vitro using any of a number of methods known in the art. Exemplary in vitro angiogenesis assays include, but are not limited to, an endothelial cell migration assay, a Matrigel tube formation assay, endothelial and tumor cell proliferation assays, apoptosis assays and aortic ring assays.

The rate of endothelial cell migration was evaluated using human umbilical vein endothelial cells (HUVEC) using a modified Boyden chamber assay as described by Clyman et al., 1994, Cell Adhes Commun. 1(4):333-42 and Lin, P et al., 1998, Cell Growth Differ. 9(1):49-58 p and further detailed below.

A matrigel tube formation assay was used to demonstrate differentiation of endothelial cells. In carrying out the assay, endothelial cells are layered on top of an extracellular matrix (Matrigel), which allows them to differentiate into tube-like structures. Angiostatin, either in the form of fusion protein or protease treated pIasminogen, has been shown to inhibit the proliferation of endothelial cells, migration of endothelial cells, inhibition of Matrigel tube formation and an induction of apoptosis of endothelial cells (O'Reily et al., Cell. 1994, 79(2):315-28 and Lucas et al., 1998, Blood 92(12):4730-41). The functionality of virally produced angiostatin in vitro was evaluated by performing endothelial tube formation assays on Matrigel, as further described below.

Endothelial and tumor cell proliferation assays were used to demonstrate the inhibitory effects of vector produced anti-angiogenic factors on cell proliferation.

An aortic ring assay was used to demonstrate the inhibition of microvessel outgrowth of rat aorta rings by virally produced angiostatin and endostatin (Kruger, E. A. et al., 2000, Biophys. Res. Comm. 268, 183-191). An aortic ring assay was used to demonstrate the inhibition of microvessel outgrowth of rat aorta rings by virally produced angiostatin and endostatin.

Angiostatin protein delivery has been shown to induce tumor apoptosis in tumor models, e.g., as described in Bergers G. et al., 1999, Science. 284(5415):808-12; Lucas R. et al., 1998. Blood. 92(12):4730-41; Kirsch M et al., 1998 Cancer Res. 15;58(20):4654-9; Claesson-Welsh L. et al., 1998, Proc Natl Acad Sci USA. 95(10):5579-83; Lannutti B J et al., 1997, Cancer Res. 57(23):5277-80.; O'Reilly M S, et al., 1996, Nat Med. 2(6):689-92; Ma H I et al., 2002, Cancer Res. 62(3):756-63; and Soff, G. A., 2000, Cancer Metastasis Rev. 19(1-2):97-107. Tumor cell apoptosis was evaluated as a further indicator of anti-angiogenic activity, as further detailed below.

V. In Vivo Evaluation of Angiogenesis

In vivo gene expression as well as the effectiveness of a given vector encoding an anti-angiogenic compound or factor may be evaluated in vivo using any of a number of methods known in the art. For example, gene expression may be evaluated by measurement of the amount of anti-angiogenic protein in the serum of animals following administration of a vector encoding an anti-angiogenic compound or factor, e.g., by immunoassays, such as ELISA (as further described below), competitive immunoassays, radioimmunoassays, Western blot, indirect immunofluorescent assays and the like. The activity, expression and/or production of mRNA for a given anti-angiogenic compound or factor may also be determined by Northern blot and/or reverse transcriptase polymerase chain reaction (RT-PCR).

Exemplary in vivo angiogenesis models include, but are not limited to, in a B16 B1/6 Mouse melanoma metastasis model (described below); a B16F10-luc metastasis model with Xenogen Imaging (described below); a Lewis Lung Carcinoma (LLC) Xenograft Resection Model (O'Reilly et al, 1994, Cell. 79(2):315-28); a LLC-luc metastasis model/Xenogen Imaging; a LLC-luc SC resection model/Xenogen Imaging; a RIP-Tag pancreatic islet carcinoma transgenic model (Hanahan et al., Nature, 315(6015):115-122, 1985 and Bergers et al., Science, 284:808-811, 1999); an orthotopic breast cancer model MDA-231 (Hiraga T. et al., 2001, Cancer Res. 61(11):4418-24); a C6 glioma model (Griscelli F, et al., 1998, Proc Natl Acad Sci USA. 95(11):6367-72) an LnCP prostate cancer model (Horoszewicz J S et al., Cancer Res. 43(4):1809-18, 1983); and a PC-3 Xenograft pancreatic tumor model (Donaldson J T et al., 1990, Int J Cancer. 46(2):238-44).

The RIP-Tag spontaneous pancreatic islet carcinoma model makes use of transgenic mice which have been genetically modified to express a rat insulin promoter (RIP) driven simian virus 40 (SV-40) antigen and develop islet cell carcinomas as a result of SV-40 oncogene expression in pancreatic islet cells. In the model, tumor development proceeds through a series of well-defined stages.

VI. Therapeutic Applications of Anti-Angiogenic Gene Therapy

The invention contemplates administration of the recombinant vectors to a patient with a tumor in order to slow or halt completely the growth of the tumor. Administration to the patient may be by any known method, including both in vivo and ex vivo modes of administration.

In vivo delivery involves delivery of a gene therapy vectors of the invention directly to a patient. In some cases, the vector is delivered to a depot organ, e.g., liver or muscle, by intraportal (IP) or intramuscular (IM) injection, respectively. In other approaches, the vector is delivered intravenously (IV). Such delivery may also be by the intraperitoneal route or by delivery directly to the tumor site. Non-invasive methods, such as oral delivery, are also contemplated. In some cases, delivery may be accomplished by an ex vivo route. Ex vivo delivery involves ex vivo (outside the body) transduction of cells by the recombinant vectors, followed by administration of the transduced cells to the patient.

The gene therapy vectors of the invention are delivered in an amount effective yield to a therapeutic level of the anti-angiogenic factor encoded by the vector in the vicinity of cancer cells or a tumor. It is preferred that the anti-angiogenic factor be present in the serum at a level of at least about 20 ng/ml and preferably at a level from about 20-800ng/ml.

The present invention contemplates treatment regimens that include the use of gene therapy vectors that encode an anti-angiogenic compound, alone or in combination with any of a number of modes of therapeutic intervention typically employed by those of skill in the art to treat cancer. In general standard therapeutic regimens for cancer treatment, including surgery, chemotherapy and radiation therapy suffer from suffer from a number of deficiencies the most important of which are a lack of efficacy and frequent toxic side effects. More recently immunotherapy methods and treatments that involve direct administration of anti-angiogenic compounds or agents are under development for cancer treatment. There remains a serious need for specific, less toxic cancer therapies. Chemotherapeutic agents for use in practicing the invention include any of a number of agents with established use in cancer therapy.

Accordingly, the present invention includes improved cancer treatment regimens that involve the use of gene therapy vectors that encode an anti-angiogenic compound, in combination with one or more of chemotherapy, radiation therapy, immunotherapy methods and treatment with a different anti-angiogenic compound (either by way of gene therapy mediated delivery or direct delivery of the compound). Examples of anti-angiogenic compounds that may be used in the treatment regimens of the present invention, include, but are not limited to, inhibitors or antibodies to vascular endothelial growth factor (VEGF) or members of the VEGF signaling pathway, inhibitors or antibodies to the epidermal growth factor (EGF) receptor; combinations of angiostatin and endostatin, soluble receptors of VEGF [soluble VEGF receptor I (sflt-1), II (sKDR) and III (sVEGFrec3)], TRAIL, thrombospondin, interferon alpha, PF-4 (platelet factor 4), PEDF and sTie2 (soluble fragment of Tie2 or Tek). From the foregoing, it can be appreciated that the compositions and methods of the present invention offer advantages in providing a means for effective and sustained delivery of an anti-angiogenic compound to a subject.

All patent and literature references cited in the present specification are hereby incorporated by reference in their entirety.

Materials and Methods

Immunoprecipitation

Western blots of virus produced Angiostatin were carried out using conditioned media, collected 48 hours after infection and heated to 60° C. for 30 min. to inactivate adenovirus. Serum was collected from mice prior to sacrifice. Both conditioned media and serum were diluted 1:10 with PBS and subjected to standard immunoprecipitation procedures using monoclonal anti-human plasminogen. Angiostatin (mouse anti-human plasminogen, Cal Biochem, San Diego, Calif.) immunoprecipitates were resolved on 10% SDS-polyacrylamide gels (acrylamide-bisacrylamide, 29:0.8). Proteins were transferred to polyvinylidene difluoride (PVDF) membranes (Immobilon-P™; Millipore, Bedford, Mass.) in transfer buffer (25 mM Tris-HCl pH 7.4, 192 mM glycine and 15% methanol) using a Trans-Blot apparatus (BioRad) for 2 h at 60 V. Protein binding sites on the membranes were blocked by incubating membranes overnight in TNT buffer [10 mM Tris-HCl pH 7.5, 100 mM sodium chloride, 0.1% (v/v) Tween 20 (Sigma)] containing 3% nonfat, powdered milk (blocking buffer). Membranes were incubated with HRP-conjugated goat anti-human plasminogen (Cedarlane Laboratories) at 0.08 mg/ml) for 1 h, washed in TNT buffer with changes every 5 min for 30 min. Human Angiostatin was detected by enhanced chemiluminescence (Amersham) according to the manufacturer's protocol.

Cultured Cells

Human umbilical vein endothelial cells (HUVEC), and Human Microvascular Endothelial cells (HMVEC) were from Clonetics (Walkersville, Md.). Primary endothelial cells were cultured in EGM-2 (Clonetics) supplemented with 20% of fetal calf serum, 1 mM glutamine, 1 mg/ml hydrocortisone, and 10 ng/ml epithelium growth factor, and infection was performed in the same medium but in 2% fetal calf serum and 3 ng/ml recombinant human basic fibroblast growth factor (R&D Systems). The multiplicity of infection (MOI) was chosen to obtain between 80% and 100% infected cells as judged by 5-bromo-4chloro-3-indolyl β-D-galactoside staining after infection with Ad CMV β-gal.

Angiostatin Antibodies and Angiostatin ELISA.

Monoclonal antibodies to human plasminogen were obtained from Calbiochem Inc (San Diego, Calif.) and used for Angiostatin immunoprecipitations. HRP conjugated goat anti-human plasminogen were used to detect human Angiostatin on Western blots. Both antibodies to human plasminogen did not cross react with mouse plasminogen. Angiostatin ELISAs were performed by using a plasminogen capture antibody and detecting antibody from Affinity Biologicals Inc (Enzyme Research Laboratories, South Bend, Ind.).

Endothelial Cell Migration Assay (Modified Boyden Chamber Migration Assay)

Briefly, a 24-well polycarbonate filter wells (Costar Transwell with an 8 um pore size) were coated with 2% gelatin in PBS for 2-4 hours at room temperature in the cell culture hood, then subsequently incubated at 37C for 1 h with DMEM containing 0.1% BSA. HUVEC cells were trypsinized, pelleted by centrifugation, washed and resuspended in fresh DMEM/BSA to a final concentration of 2×10⁶ cells /ml. Aliquots of cells 2×10⁵ cells were applied to the upper chamber of the filter wells. The filter inserts with cells were placed in wells of a 24-well culture plate containing either media alone as a control, or media plus human recombinant VEGF or bFGF at 10 ng/ml preincubated for 30 min with conditioned media transduced with adenovirus vectors encoding angiostatin or GFP/lacZ at an MOI of 1-10 and diluted to contain approximately 300 ng/ml of angiostatin. After a 6 hour incubation at 37C, the cells that have migrated to the lower surface of the filter inserts were fixed with Diff-Quik (Dade International), fixed for 2 min; solution I for 2 min and solution II for 3 min. Filter inserts were examined under a microscope at 200× magnification.

Matrigel Tube Formation Assay

Matrigel (Beckton Dickinson) was coated onto 24-well cell culture plates on ice, and incubated at 37C for 30 min. Conditioned medium from adenovirus transduced cells were collected and assayed for Angiostatin production. Conditioned medium was then titrated to contain 300 ng/ml of Angiostatin and used to layer on top of the matrigel coated plates. 5×10⁵ HUVEC cells were added on top of the conditioned media. Plates were incubated for 12 hours at 37C, and plates were scored by the total no of junctions formed by the endothelial cells from 5 fields and averaged under the microscope.

Endothelial And Tumor Cell Proliferation Assays

HUVEC cells were infected with candidate viral vectors, e.g., adenovirus encoding Angiostatin or lacz at an MOI of 0.1, 1, 10, 100 and collected with 1 mM EDTA, washed twice with PBS, and resuspended. They were seeded into 96-well cell culture plates at 5000 cells/well and cultured for 12 hr, followed by addition of BrdU. DNA synthesis was measured by BrdU incorporation into the cells with a BrdU incorporation assay kit (Roche, Mannheim, Germany).

Cell Cycle Analysis

HUVEC cells were infected with viral vectors, e.g., adenovirus encoding Angiostatin or lacz at an MOI of 0.1-100 and incubated at 37° C. Cells were harvested after 12-72 hrs by treatment with cell detachment buffer (Sigma) for 2 min and subsequent washes/spins for 2 times. Cells were resuspended into EGM-2 media and treated with propidium iodide in solubilization buffer (Triton, RNAse, etc.) and DNA content was analyzed by FACS analysis (Becton Dickinson).

Apoptosis Assays

HUVEC cells were infected with adenoviruses encoding Angiostatin or lacz at an MOI of 0.1, 1, 10, 100 and collected with 1 mM EDTA, washed twice with PBS, and resuspended. They were seeded into 96-well cell culture plates at 5000 cells/well and cultured at 37 C. Apoptosis assays were performed on the supernatant of these cells by using the Cell Death Detection ELISA^{plus} kit from Roche (Mannheim, Germany) which is a photometric enzyme-immunoassay that measures cytoplasmic histone-associated-DNA-fragments after induced cell death.

Aortic Ring Assay

12-well tissue culture plates were covered with Matrigel (Becton-Dickinson, Bedford, Mass.) and allowed to solidify for 1 hours at 37C incubator. Thoracic aortas were excised from 4-6 week old male Sprague-Dawley rats and the fibroadipose tissue was removed. Aortas were sectioned into 1.2 mm long cross sections. Rinsed numerous times with EGM-2 (Clonetics Inc.), placed on Matrigel coated wells, and covered with additional Matrigel then allowed to solidify at 37° C. for another hour. The rings were cultured overnight in 2 ml of EGM-2, the next day the media was removed, and the rings were cultured with bFGF and virally produced angiostatin, endostatin, or recombinant angiostatin/endostatin for 4 days.

Histology

Lung and liver tissues were harvested after tumor assessment and flash frozen immediately. 8 um sections were cut and stained with hemotoxylin and eosin. TUNEL staining was performed by using In Situ Cell Death Kit (Roche).

Liver Enzyme sGPT Analysis

Serum samples were collected from animals before and after various time points after gene delivery. Serum glutamine pyruvate transaminase (sGFI) assays were performed using kits obtained from Sigma (St. Louis, Mo.). The values are expressed in international units/ml.

B16 B1/6 Mouse Melanoma Metastasis Model

Female C57B/6 mice were obtained from Taconic and mice were at 6-8 weeks old at the start of each experiment. Mice were injected with 5×10⁴ B16B1/6 cells on day 0 via tail vein with a 27-gauge needle. After 14-21 days, mice were sacrificed and their tumor burden assessed by harvesting the mice lungs and counting the surface tumor metastasis and measuring the weight of the lung. All experiments had 6-10 animals per group. Statistical significance was evaluated using the Student's t- test.

Xenogen Imaging of Tumor Models

In vivo luminescence of tumor bearing mice were monitored by biweekly monitoring of B16F10-luciferase (Xenogen Inc.) injected mice. In brief, Balb/c nu/nu mice were injected with 5×10⁴ or 2×10⁵ cells of B16F10-luc cells via tail vein on day 0. Mice were monitored for tumor burden when necessary by intra-peritoneal injection of excess luciferin substrate at 1.5 mg/g mice weight. Twenty minutes after substrate injection, mice are anesthesized and monitored for in vivo luminescence with Xenogen IVIS Imaging System (Xenogen Inc.) luminescence sensitive CCD camera by dorsal or ventral position. Data is collected and analyzed by Living Image 2.11 software. CCD photon counts were analyzed by Living Image 2.11 and an Excel spreadsheet.

The following examples illustrate but are not intended in any way to limit the invention:

### EXAMPLE 1

### Construction and Characterization of Recombinant Adenoviral (AV) vectors Encoding Angiostatin and Endostatin.

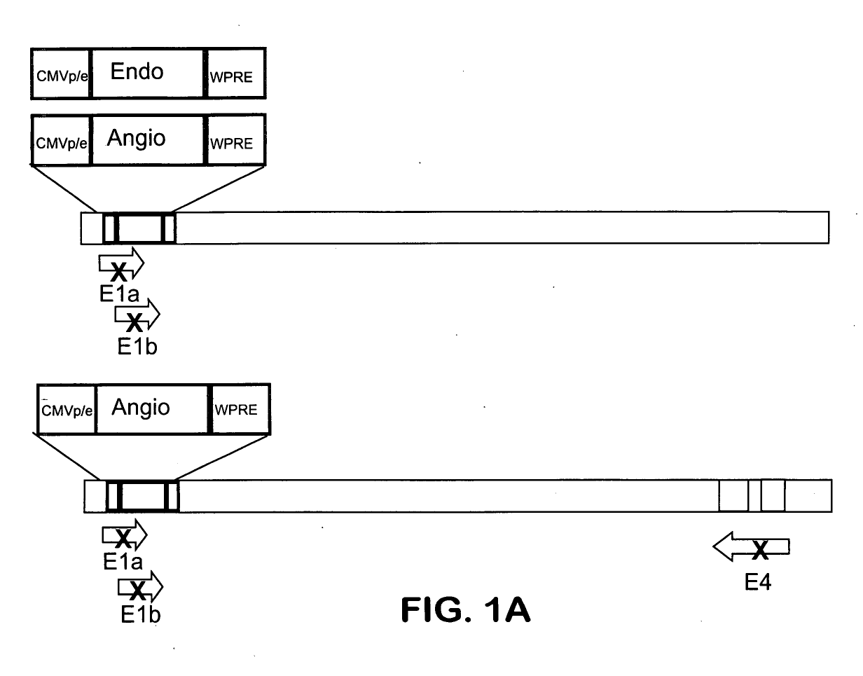

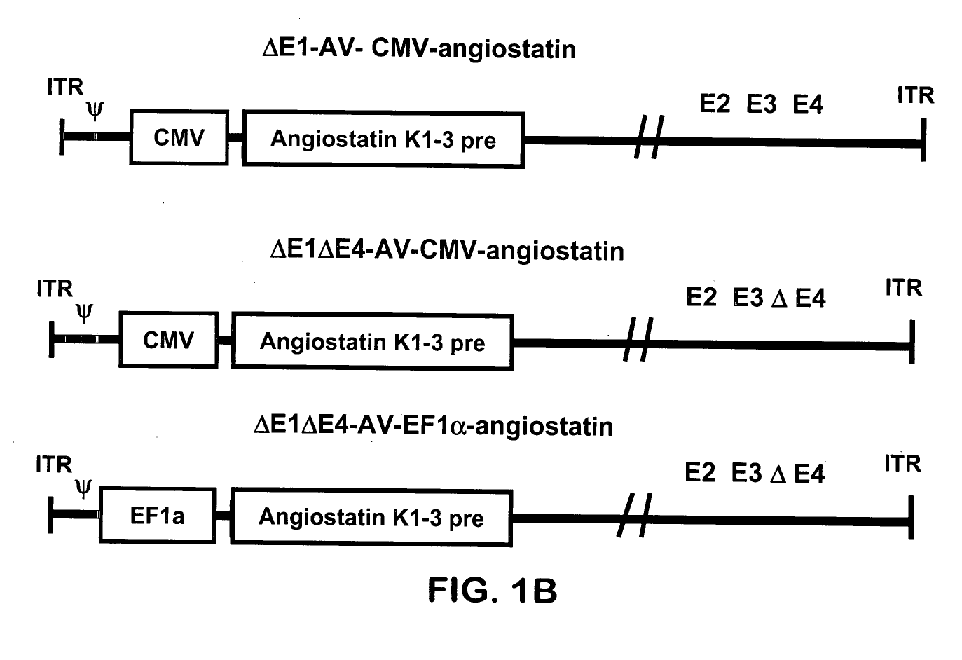

Recombinant AV vectors encoding human angiostatin and endostatin polypeptides were constructed by standard techniques. One set of vectors were E1a and E1b deleted and another vector, expressing angiostatin, was E1a/E1b/E4 deleted (FIGS. 1A and B).

### ΔE1, ΔE1/ΔE4 adenoviruses encoding angiostatin K1-3

One example of vector construction employed homologous recombination techniques in yeast and DNA sequences encoding human plasminogen K1-3 (angiostatin) was introduced into the E1 region of either E1-deleted (ΔE1-AV) or E1E4 deleted adenoviral vector (ΔE1/ΔE4-AV).

More specifically, the following primers were designed for the amplification of the angiostatin transgene:

(Angio Forward, SEQ ID NO: 8)

5′-CGTACCTAGGGAATTCAAGCTTACTAGTGCCGCCGCCATGGATGCAA
TGAAG AGAGG 3′; and

(Angio Reverse, SEQ ID NO: 9)

5′ GTCAGTCGACTCTAGAAGTGGATCTTTAAGGTGGTGCTG 3′.

These primers contain several restriction enzyme sites with Avr II and Sal I designed for the 5′ and 3′ end of the amplicon to allow for directional cloning of the transgene. PCR reactions were set up with the Pwo DNA Polymerase Kit (Roche Molecular Biochemicals) under the following conditions: 94° C. for 4 minutes, 5 cycles of 94° C. for 1 minute, 55° C. for 1 minute, and 68° C. for 3 minutes, and 15 cycles of 94° C. for 1 minute, 63° C. for 1 minute, and 68° C. for 3 minutes. The backbone, ploxCMVpre contains 0-15.8 m.u. of AD5, the CMV promoter including the CMV splice donor intro paired with the alpha-globin splice acceptor, an Avr II /Sal I polylinker, the Woodchuck hepatitis virus post-transcriptional regulatory element (WPRE), and SV40 poly(A). Yeast strain YPH857 was transformed with the AD5 PAC backbone and the CMV angiostatin vector by the lithium acetate method and standard screening protocols used. (ref) The ΔE1 CMV angiostatin and the ΔE1/E4 angiostatin PACs were grown in bacteria and PAC DNA was isolated using a Qiagen Maxi prep kit. The PACs were cut with SCE I Meganuclease to release the adenovirus from the PAC backbone and transfected into 293 based complimentary cell lines. Recombinant adenoviruses were isolated onto 293-derived cell monolayers and amplified onto fresh 293 cells, with viral stocks were prepared as previously described (Graham, F L and Prevec, L, 1995, Nat. Biotechnol. 3:207-220.). Ad ΔE1 CMV GFP, lacZ and the ΔE1/E4 GFP and lacZ were prepared in the same manner as the angiostatin adenoviral vectors.(Patel, S D et al., 2000, Mol. Ther. 2:161-169). Recombinant adenovirus was generated and purified by standard methods (Graham, F L and Prevec, L, 1995, Nat. Biotechnol. 3:207-220). Adenovirus were titered by measuring A260 and the results converted into virus particles (vp). Hexon assays were performed on either 293 cells for the Ad ΔE1 viruses or 293-Orf6 producer cell (or MIP-56) with IMX induction for the Ad ΔE1/E4 viruses (Wang, Q Jia, X C and Finer, M H, 1995, Gene Ther. 2:775-783).

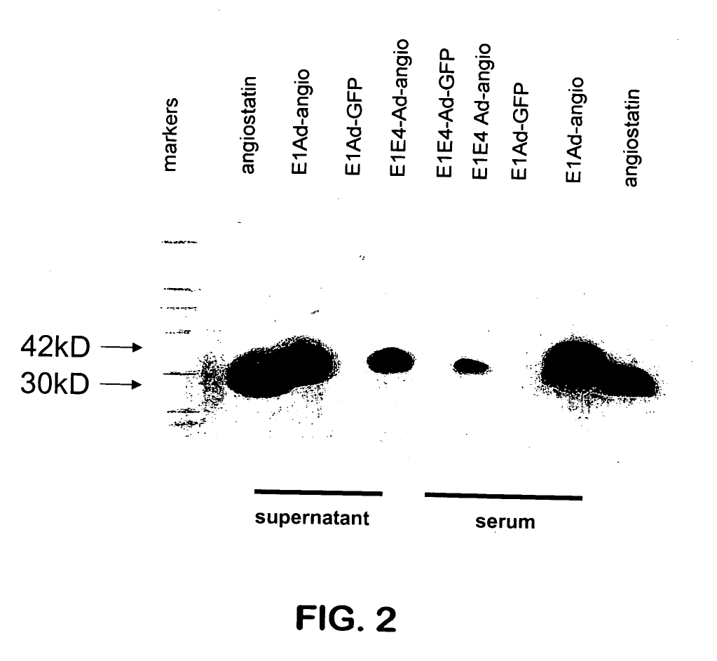

Adenoviruses encoding angiostatin were generated after transfection of the two vector DNAs into 293 cells as described. In vitro assays were used to evaluate the expression of the recombinant gene therapy vectors. 293 cells were transduced with ΔE1-AV-angiostatin, ΔE1/ΔE4-AV-angiostatin, with conditioned media collected. A combination of immunoprecipitation and Western-blotting confirmed the presence of angiostatin expression from ΔE1-AV-angiostatin, ΔE1/ΔE4-AV-angiostatin (FIG. 2). ΔE1-AV-angiostatin, ΔE1/ΔE4-AV-angiostatin and GFP control virus were used to transduce 293 cells with a similar MOI. Conditioned media was collected 48 hrs later and subjected to immunoprecipitation-Western blotting with anti-human plasminogen. Angiostatin produced from both adenovirus infection of 293 cells produced a protein that cross reacted with anti-human plasminogen with an apparent molecular weight of 37 kD. This angiostatin produced from 293 cells had a slower mobility than the angiostatin K1-3 purified from Pichia due to the presence of an intact N-linked glycosylation site (N306) in the kringle 3 region of plasminogen. This site has been shown to be N-glycosylated in both human and mouse plasminogen (Pirie-Shepherd, S. R et al., 1997, J. Biol. Chem. 272 (11), 7408-7411).

All of the angiostatin and endostatin vectors tested expressed angiostatin or endostatin proteins, respectively, in vitro. AAV vectors encoding angiostatin alone or angiostatin and endostatin in combination also expressed the appropriate sized product from cells transduced in vitro. The relative levels of angiostatin and endostatin expressed by cells transduced in vitro with various vectors is summarized in Table 1. The summary of the characterization performed on angiostatin and endostatin produced by these vectors is shown in Table 2.

TABLE 1
Angiostatin And Endostatin Expressed By Cells Transduced In Vitro

| | Endostatin | Angiostatin |
|---|---|---|
| | ng/ml/1.2 × 10⁶ | ng/ml/1.2 × 10⁶ |
| Vector | 293 cells | 293 cells |
| AAV endo pre1 | 197 | N/A |
| AAV endo pre2 | 310 | N/A |
| AAV angio pre1 | N/A | 2846 |
| AAV angio pre2 | N/A | 3363 |
| AAV EF1-α angio | N/A | 10324 |
| AAV ires1^{#} | 81 | 775 |
| AAV ires2^{#} | 73 | 489 |
| AAV ires1n | — | 1500 |
| AAV ires2n | — | 1379 |
| AAV tr1* | 206 | 110 |
| AAV tr2* | 435 | 165 |
| AV E1 endo pre | 3938 | — |
| AV E1 angio pre | — | 4200 |
| AV E1E4 angio pre | — | 4100 |

TABLE 2
Characterization Of Angiostatin And Endostatin Expressed In Vitro

| Construct | Western | ELISA | IP/Western |
|---|---|---|---|
| AAV − E | + | + | + |
| AAV − A | + | + | + |
| AAV − A(IRES)E | +/+ | +/+ | +/+ |
| AAV − A + E | +/+ | +/+ | +/+ |
| ΔE1 − AV − E | + | + | + |
| ΔE1 − AV − A | + | + | + |
| ΔE1 − AV − E + A | ND | ND | ND |
| ΔE1/ΔE4 − AV − A | + | + | + |
| ΔE1/ΔE4 − AV − E | − | ND | ND |
| ΔE1/ΔE4 − AV − E + A | − | ND | ND |

### EXAMPLE 2

### In Vitro Evaluation of Recombinant Adenoviral (AV) vectors Encoding Anti-angiogenic Compounds

The functionality of our adenoviral produced angiostatin was evaluated in vitro using endothelial migration assays and proliferation assays.

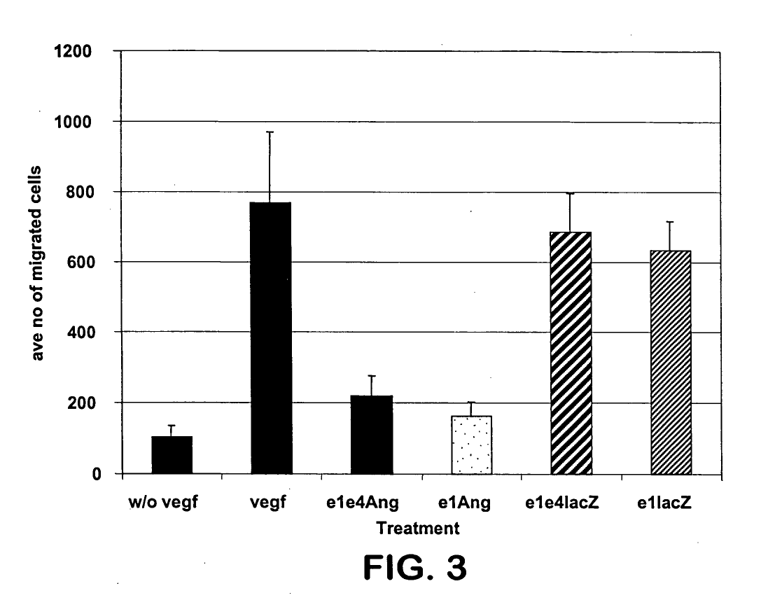

Both ΔE1-AV, ΔE1/ΔE4-AV encoding angiostatin and lac-Z were used to infect HUVEC cells at an MOI of 10. After 48 hrs of transduction, conditioned cell culture media was collected and heat inactivated to kill residual virus and then used in a modified Boyden chamber to assess its effect on HUVEC migration. The conditioned media from the two different virus-infected 293 had similar levels of angiostatin when assayed by ELISA (data not shown). The conditioned media collected from ΔE1-AV-angiostatin, ΔE1/ΔE4-AV-angiostatin transduced cells and control lacZ conditioned media were diluted to contain an identical concentration of angiostatin. Endothelial cells were incubated with conditioned media from ΔE1-AV-angiostatin, ΔE1/ΔE4-AV-angiostatin or ΔE1-AV-lacZ, ΔE1/ΔE4-AV lac Z infected cells and assayed for their ability to migrate to the lower chamber that contained stimulants such as VEGF (FIG. 3). Both ΔE1-AV-angiostatin, ΔE1/ΔE4-AV-angiostatin inhibited HUVEC cell migration at 0.3 μg/ml whereas the control groups AV encoding lacZ culture media did not (FIG. 3).

Endotheial cells transduced with either ΔE1/ΔE4-AV-angiostatin or ΔE1/ΔE4-AV-lacZ were also subjected to proliferation assays as another measure for the functionality of virally produced angiostatin. BrdU was added to cells expressing angiostatin or lacZ and BrdU incorporation measured as a readout for new DNA synthesis. Transduction of HUVEC by ΔE1/ΔE4-AV-angiostatin resulted in an inhibition of cell proliferation compared to ΔE1/ΔE4-AV- lacZ transduced cells at an MOI of 1. Seventy-two hours after virus transduction, ΔE1I/ΔE4-AV-angiostatin inhibited HUVEC DNA synthesis by 74% compared to the ΔE1/ΔE4-AV- lacZ infected cells. However, ΔE1/ΔE4-AV-angiostatin did not inhibit the proliferation of B16B1/6 mouse melanoma cells, or HepG2 human hepatoma cells (data not shown). In addition, a dose dependent inhibition of DNA synthesis was achieved when the MOI was increased. A logarithmic incremental increase of MOI from 0.01 to 1 correlated with the decrease in the DNA synthesis by HUVEC cells, indicating a dose-dependent effect.

The proteins encoded by the vectors were also shown to be bioactive in a series of in vitro cell based assays. Endostatin expressed an ΔE1-AV vector inhibited the migration of endothelia cells in a Boyden chamber assay.

Angiostatin expressed from ΔE1-AV vectors was biologically active in inhibiting the proliferation of endothelial cells. In a FACS based assay, ΔE1-AV-angio transduced endothelial cells were shown to be arrested in S phase (92%) compared to AV-GFP transduced cells (14%).

Angiostatin has been shown to induce apoptosis of endothelial cells (Lucas et al., 1998, Blood. 92(12):4730-41). A study was carried out to determine if adenovirus produced angiostatin had an effect on the rate of apoptosis of endothelial cells. To determine if adenovirus produced angiostatin had an effect on the rate of apoptosis of endothelial cells, the amount of DNA fragmentation in either angiostatin or lacZ transduced HUVEC cells was determined after they were lysed in situ.

HUVEC cells were transduced at an MOI of 1 with either ΔE1/ΔE4-AV-angiostatin or ΔE1/ΔE4-AV-lacZ, and further assayed for DNA fragmentation as measure of the degree of apoptosis. Increased DNA fragmentation of HUVEC was demonstrated 48 hrs after transduction with ΔE1/ΔE4-AV-angiostatin compared to ΔE1/ΔE4-AV-lacZ at an MOI of 1. After 48 hrs, ΔE1/ΔE4-AV-angiostatin transduced cells were expressing angiostatin at 1000 ng/ml. The increase in apoptosis was dependent upon the concentration of virus used, with a higher MOL ΔE1/ΔE4-AV-angiostatin resulting in a significant increase in DNA fragmentation of HUVEC cells (data not shown). This increase in MOI also correlated with an increase in angiostatin in the cell culture media. These results show that adenovirus produced angiostatin inhibits HUVEC proliferation, as well as increasing the rate of apoptosis.

### EXAMPLE 3

### In vivo Evaluation of Recombinant Adenoviral (AV) Vectors Encoding Anti-Angiogenic Compounds.

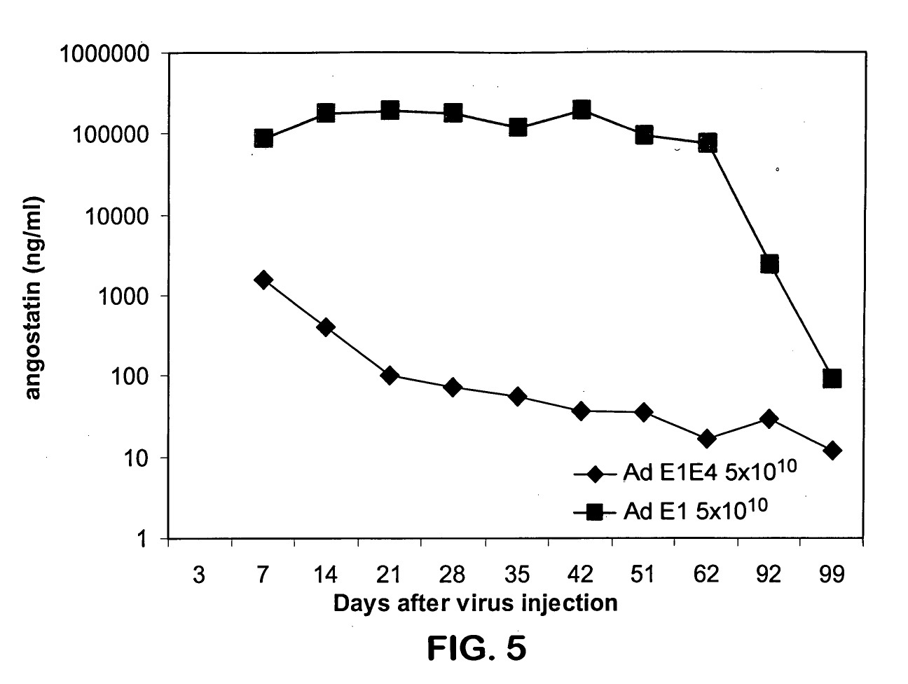

ΔE1-AV-angiostatin, ΔE1/ΔE4-AV-angiostatin and GFP control virus were injected into mice at 5×10¹⁰ vp and serum was collected 48 hrs after and subjected to immunoprecipitation and Western blotting. The angiostatin taken from mouse serum had the same mobility and apparent molecular weight as angiostatin from conditioned media. These results showed that angiostatin produced in vivo using adenoviral vectors was shown to be identical in size to in vitro produced angiostatin. Expression levels of angiostatin were evaluated by angiostatin ELISA, the result of which are presented in FIG. 5.

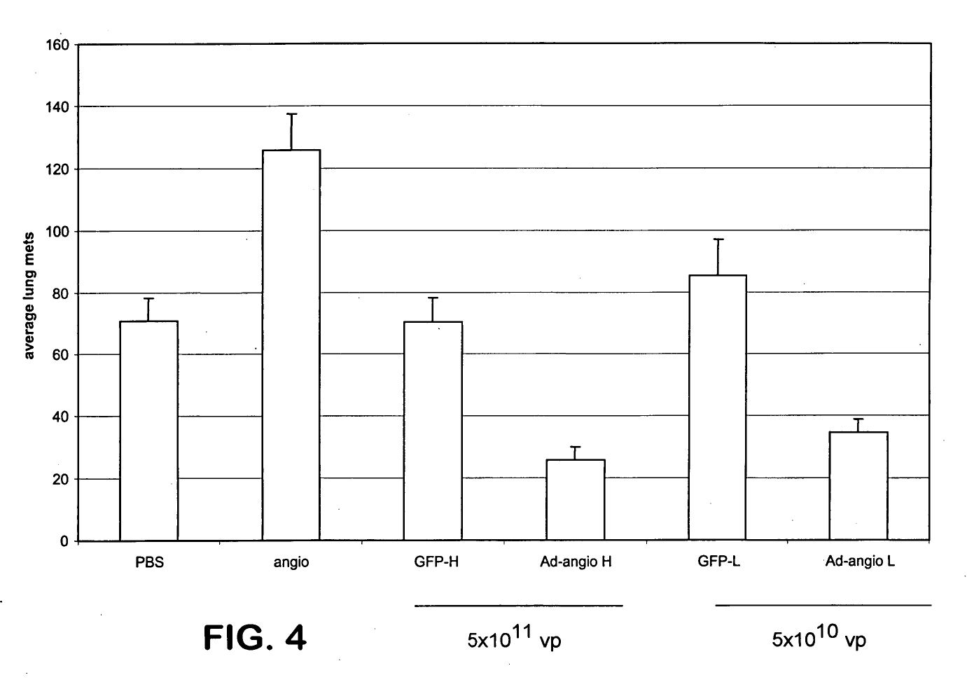

The anti-tumor efficacy of ΔE1-AV-angiostatin and ΔE1/ΔE4-AV-angiostatin, was further tested in a B16B1/6 murine melanoma metastasis model. Mouse melanoma cells were injected via tail vein into syngeneic 5-6 week old C57B1/6 mice on Day 1, and Two days later the mice were treated with either daily injections of endostatin or angiostatin or a single injection of 10¹⁰ vp or 10¹¹ vp of one of the following vectors: ΔE1-AV-Angio, ΔE1-AV-Endo or ΔE1-AV-GFP. On Day 15, mice were sacrificed and tumor assessments were made by counting surface lung metastases. Injection of 1010 virus particles, both ΔE1-AV-angiostatin and ΔE1/ΔE4-AV-angiostatin, inhibited tumor metastasis by 70% when compared to the ΔE1-AV-GFP, ΔE1/ΔE4-AV-GFP controls or the PBS injected control groups of mice (FIG. 4). Mice serum was collected prior to virus injection, and at two time points after virus injections. Angiostatin ELISAs revealed the in vivo steady state expression of angiostatin two days and thirteen days after virus injection. Two days after the ΔE1-AV-angiostatin and ΔE1/ΔE4-AV-angiostatin injections in mice, we detected 4.1 μg/ml and 0.33 μg/ml of angiostatin in mice serum respectively (FIG. 5).

A second generation E1/E4 deleted AV vector encoding angiostatin was created based on published observations that E1/E4 deleted adenovirus exhibits less toxicity than ΔE1 adenovirus (Wang Q, Finer M H., 1996, Nat Med. 2(6):714-6). ΔE1/ΔE4-AV-angiostatin was delivered intravenously and compared to ΔE1-AV-angiostatin for its effect on the transduced liver of mice. The liver toxicity of the first generation adenoviral vectors had been correlated with elevated serum glutamic pyruvic transaminase (sGPT) levels and heavy neutrophil infiltration into the liver parenchyma. The results showed that intravenous delivery of ΔE1/ΔE4-AV-angiostatin at 10¹⁰ vp and 10¹¹ vp resulted in serum sGPT levels that were well within the normal range following virus injection when liver toxicity has shown to be close to its peak (Wang Q Finer M H., 1996). Creatinine levels were also monitored and remained within normal range for both ΔE1/ΔE4-AV-angiostatin and ΔE1-AV-angiostatin at two doses (Brody S L and Crystal R G., 1994, Ann N Y Acad Sci. 716:90-101). Liver H&E histology confirmed indicated that the transduced hepatocytes appeared normal after intravenous delivery of 10¹⁰ or 10¹¹ vp of ΔE1/ΔE4-AV-angiostatin. This was in contrast to ΔE1-AV-angiostatin, which resulted in elevated liver sGPT following intravenous delivery of 10¹⁰ vp of ΔE1-AV-angiostatin.

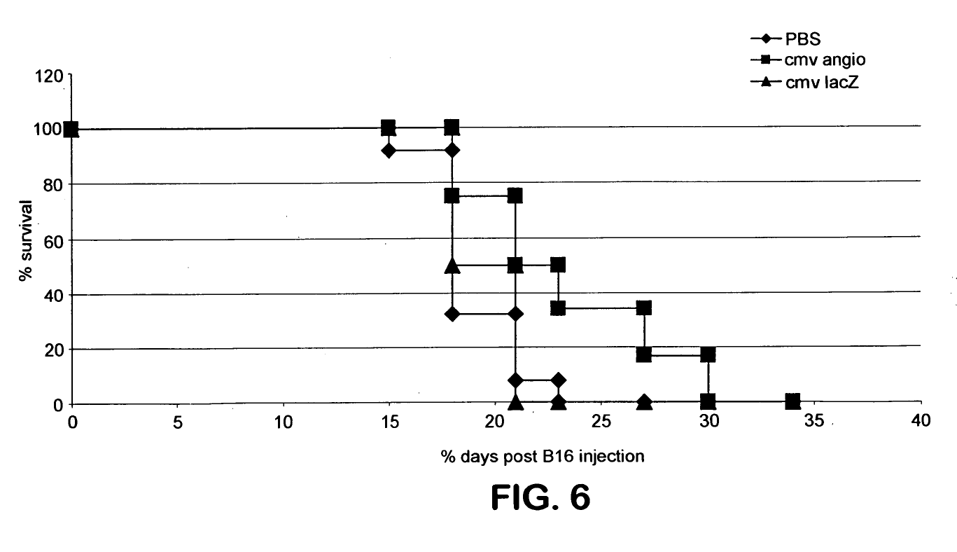

One of the major limitations of adenovirus gene therapy is reported to be that transgene expression in vivo is transient. To determine the d uration of expression from our gene therapy vectors over time, ΔE1-AV-angiostatin or ΔE1/ΔE4-AV-angiostatin was injected intravenously into immunocompetent C57B1/6 mice at 5×10¹ vp on Day 1. Serum angiostatin levels were monitored weekly for approximately 14 weeks. The results demonstrate that angiostatin expression in the mice was sustained at more than 50 ng/ml for over 8 weeks. At day 99, mice injected with either ΔE1-AV-angiostatin or ΔE1/ΔE4-AV-angiostatin were still producing detectable amounts of angiostatin in their bloodstream (FIG. 5). Mice that were injected with adenovirus angiostatin vectors that were sacrificed on Day 120 were still healthy and showed no signs of abnormality after extensive biopsies. Both ΔE1-AV-angiostatin, ΔE1/ΔE4-AV-angiostatin delivered mice were also assayed for their sGPT and creatinine over the course of 99 days. The survival of mice in the B16B1/6 tumor model was evaluated from 0 to 34 days following intravenous injection of 5×10¹⁰ vp of ΔE1/ΔE4-AV angiostatin or ΔE1/ΔE4-AV LacZ where the gene was expressed under control of the CMV promoter. Survival data for these mice is presented in FIG. 6.

The results presented above show that the two adenoviral vectors ΔE1 and ΔE1/ΔE4 adenovirus encoding angiostatin express and secrete angiostatin in vitro as well as in vivo in mice. The results also demonstrate the anti-tumor efficacy following systemic delivery of angiostatin by ΔE1-AV-angiostatin or ΔE1/ΔE4-AV-angiostatin in the B16B1/6 murine metastasis model.

### EXAMPLE 4

### Construction of Recombinant AAV Vectors Encoding Endostatin and Angiostatin

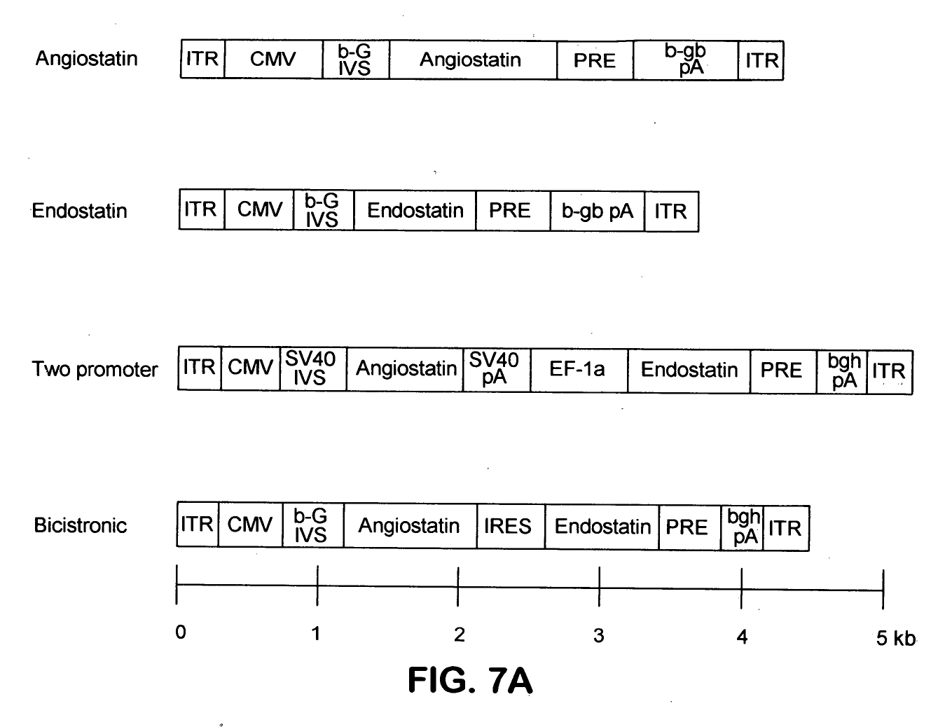

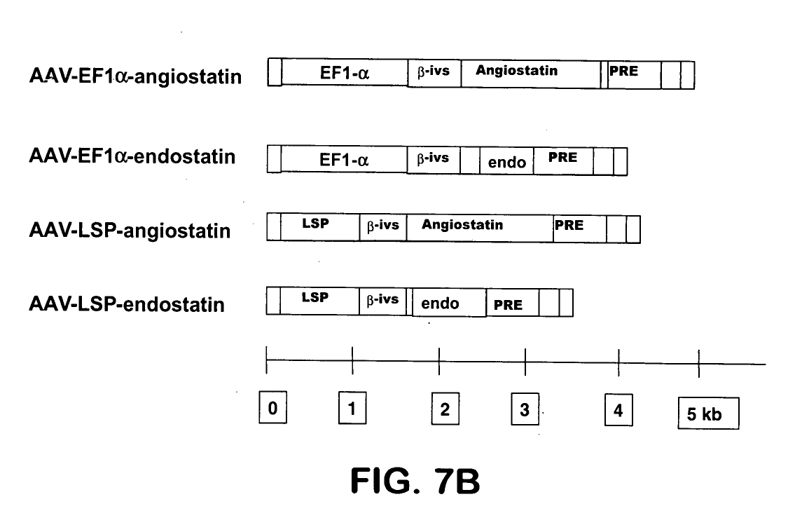

The recombinant AAV vectors encoding human angiostatin and endostatin polypeptides as set forth in FIGS. 7A and B were constructed by standard techniques. The following abbreviations are used:

ITR—the AAV inverted terminal repeat sequences

CMV—the human cytomegalovirus immediate-early (IE) promoter

beta, β, beta-G or beta-gb—human beta-globin

IVS—intervening sequence (intron)

Angiostatin—a nucleotide sequence encoding a human angiostatin polypeptide sequence consisting of the amino acids Val₇₉ through VaL₃₃₈ of human plasminogen, wherein Asn289 has been changed to Glu289.

Angiostatin K1-3—kringle domains 1-3 of human angiostatin

Endo—a nucleotide sequence encoding the following human endostatin polypeptide sequence (SEQ ID NO:11, encoded by the nucleotide sequence found at GenBank Accession No:NM_{—}130445, SEQUENCE ID NO:10):

HSHRD FQPVL HLVAL NSPLS GGMRG IRGAD FQCFQ QARAV

GLAGT FRAFL SSRLQ DLYSI VRRAD RAAVP IVNLK DELLF

PSWEA LFSGS EGPLK PGARI FSFDG KDVLR HPTWP QKSVW

HGSDP NGRRL TESYC ETWRT EAPSA TGQAS SLLGG RLLGQ

SAASC HHAYI VLCIE NSFMT ASK.

WPRE or PRE- the woodchuck hepatitis virus post-transcriptional regulatory element

pA—polyadenylation sequence

EF-1-alpha—the elongation factor EF-1-alpha promoter region

IRES—internal ribosomal initiation site sequence

The DNA sequence of human angiostatin K1-3 along with its own natural human plasminogen signal sequence was PCR amplified from human plasminogen K1-3 (angiostatin) cDNA plasmid pcDNA3.1/hAS1-3 (MacDonald et al., 1999, Biochem Biophys Res Commun. 264(2):469-77) and introduced into the AAV vector plasmid pSSV9MD2 along with the Woodchuck hepatitis virus posttranscriptional regulatory element (WPRE: Zufferey, 1999, J Virol. 73(4):2886-92) to create pSSV9MD2 CMVangio-pre) and introduced into the AAV vector plasmid pSSV9MD2 along with the Woodchuck hepatitis virus posttranscriptional regulatory element (WPRE) to create pSSV9MD2 CMVangio-pre. The vector plasmid pSSV9-MD2-angioPre was constructed by digesting plasmid pSSV9-MD2 with EcoRI and inserting a 1599 bp fragment containing the angiostatin gene derived from pcDNA3.1/hAS1-3 (MacDonald N J et al., 1999, Biochem Biophys Res Commun. Oct. 22, 1999;264(2):469-77) and the_Woodchuck hepatitis virus posttranscriptional regulatory element (WPRE). The MD2 expression cassette consists of the cytomegalovirus immediate early promoter/enhancer and the intervening sequence 2 and polyadenlyation signals from the human p-globin gene. The resulting vector genome from ITR to ITR is 4013 bp. Subsequently, the EF1-α [symbol] promoter and LSP promoter were subdoned into the pSSV9 MD2 vector to replace the CMV promoter in the parental plasmid pSSV9 CMV angio pre, thus creating pSSV9-MD2-EF1-α [symbol] angiostatin pre, and pSSV9-MD2-LSP angiostatin pre.

Vector plasmid pTR-CMV-angio-EF1α-endoPRE was generated from plasmid pTR-UF5. The GFP gene was replaced with the angiostatin gene putting it under the control of the CMV promoter and SV40 splice donor/splice acceptor region 5′ to the gene and the SV40 polyadenylation signal 3′ to the gene. The TK promoter and neo gene of pTR-UF5 were replaced with the EF1α promoter derived from plasmid pEF-Bos, the endostatin gene from pcDNA3.1/hES (MacDonald N J et al., 1999) and the WPRE. The bovine growth hormone polyadenylation signal and 3′ AAV_ITR from pTR-UF5 were retained. The entire vector genome was 5189 bp.

AAV vectors were prepared according to Snyder et al., 1997, Nature Genetics 16(3):270-6. Briefly, subconfluent 293 cells were cotransfected with a vector plasmid and the helper plasmids pUC-ACG using the calcium phosphate method. Cells were then infected with adenovirus Ad5 dl312 (an E1A⁻ mutant) at an MOI of from 2-5 and the infection was allowed to proceed for 72 hr. Cells were harvested and lysed by three freeze/thaw cycles. Lysates were treated with benzonase (Sigma) and then centrifuged to remove the cellular debris. The cleared cell lysate was fractionated by ammonium sulfate precipitation and the rAAV virions were isolated on two sequential CsCl gradients. The gradient fractions containing rAAV were dialyzed against sterile PBS containing 0.9 mM CaCl and 0.5 mM MgCk, heated for 30 min at 56° C. to inactivate any residual adenovirus. Viral titers were determined by dot-blot analysis and represent DNAse resistant genome equivalents per ml. Vector preparations were typically 1012 vp/ml.

### EXAMPLE 5

### In Vitro Evaluation of Recombinant AAV vectors Encoding Angiostatin and Endostatin

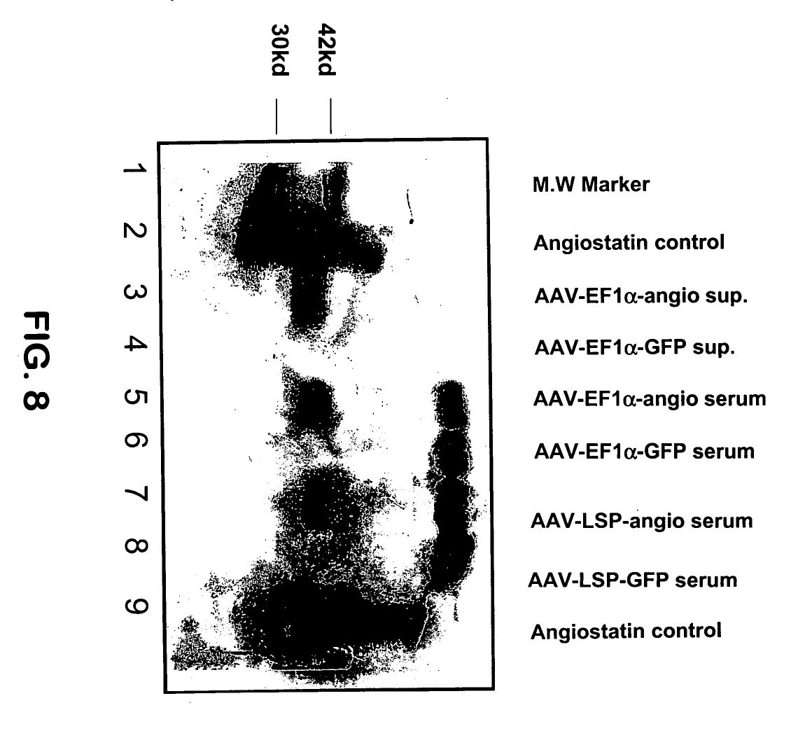

In vitro biochemical assays were used to evaluate the expression of the rAAV vectors. 293 cells were transduced with rAAV vectors with EF-1-α-angiostatin or EF-1-α-GFP at 10⁵ vp/cell and conditioned media was collected 48 hrs later. A combination of immunoprecipitation and Western-blotting was carried out as described above. The results confirmed angiostatin expression from AAV-angiostatin vectors and not the AAV-GFP control (FIG. 8). Angiostatin produced from AAV-angio infection of 293 cells cross reacted with anti-human plasminogen with an apparent molecular weight of 37 kD. This angiostatin produced from 293 cells had a slower mobility than the angiostatin K1-3 purified from Pichia presumably due to the presence of an intact N-linked glycosylation site (N306) in the kringle 3 region of plasminogen.

In addition, serum collected from mice that were injected intraportally with AAV EF1-α angiostatin and LSP angiostatin was subjected to angiostatin immunoprecipitation-Western blotting. Angiostatin circulating in mice serum had the same mobility and apparent molecular weight as angiostatin from conditioned media. In summary, all of the angiostatin and endostatin vectors tested were shown to express angiostatin and endostatin proteins in vitro. In vivo, AAV- EF1-α [symbol] angiostatin and AAV-LSP angiostatin produced similar amounts of angiostatin when both vectors were injected intraportally into Balb/c nu/nu mice at 3×10¹¹ vp/mouse.

Both angiostatin Western blots and angiostatin ELISAs confirmed the secretion of angiostatin from AAV vectors in vitro.

293 cells were transduced with AAV EF1α-angiostatin or AAV EF-1-α-GFP at 10⁵ vp/cell (MOI=10⁵). Conditioned cell culture media was collected and assayed for human angiostatin concentration. Conditioned medium collected from AAV-angiostatin and control AAV-GFP infected cells were diluted with media to contain an identical concentration (0.5 [symbol] g/ml) to Pichia recombinant Angiostatin K1-3. Endothelial cells were incubated with conditioned media from AAV-GFP and angiostatin infected cells and assayed for their ability to inhibit endothelial cell differentiation into tube-like structures when layered on Matrigel.

Both recombinant angiostatin and AAV produced-angiostatin conditioned media inhibited HUVEC tube formation at 0.5 μg/ml while AAV-GFP culture media and mock media alone did not. In addition, increasing amounts of AAV produced angiostatin correlated with increased inhibition of Matrigel tube formation in an assay where endothelial cells are layered on top of an extracellular matrix (Matrigel) which allows them to differentiate into tube-like structures.

### EXAMPLE 6

### In Vivo Evaluation of Recombinant AAV vectors Encoding Angiostatin and Endostatin

A. Evaluation of Serum Angiostatin Levels

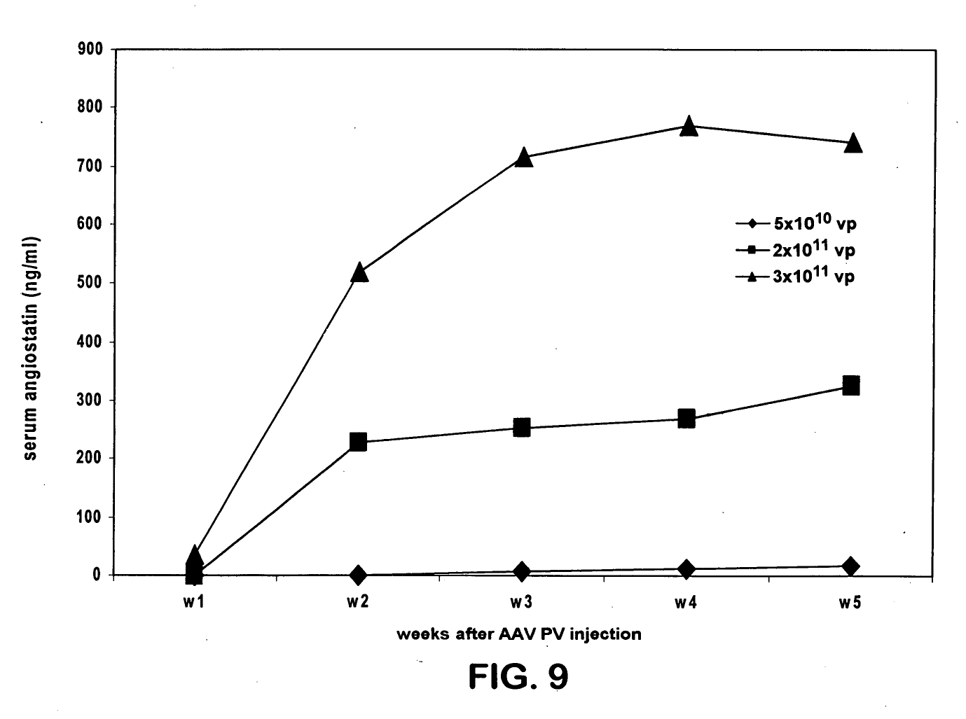

AAV gene therapy is reported to provide for sustained long-term expression of transgenes in vivo. To determine the duration of the duration of angiostatin expression in mice serum, AAV-angiostatin was injected into the immunocompromised Balb/c nude mice at 211 vp via the portal vein route and the intrasplenic route on Day 1. These two routes of virus delivery mainly target the liver. The serum angiostatin concentration was stablilized around week 5 and continued to express angiostatin until at least week 11. The effect of virus dose on angiostation expression was also evaluated. Three doses of rAAV EF1-aangiostatin (5×10¹⁰ vp, 2×10¹¹ vp, and 3×10¹¹ vp) were injected into mice via portal vein delivery and evaluated for serum angiostatin concentration over 5 weeks (FIG. 9A). In a follow-up experiment with an EF1a driven angiostatin cassette, levels of 20 to 80 ng angiostatin/ml sera were observed in mice that received a single injection of 2×10¹⁰ vp of the AAV-Angio. To evaluate AAV vectors for the ability to express recombinant angiostatin in vivo, Balb/c nu/nu mice were injected with 5×10¹⁰ vp, 2×10¹¹ vp or 3×10¹¹ AAV EF1α-angiostatin viral particles via the portal vein (pv) and serum angiostatin levels were evaluated using an angiostatin ELISA Assay. The results shown in FIG. 9 indicate that levels as high as 700 ng/ml can be achieved in vivo following administration of 3×10¹¹ AAV EF1α-angiostatin viral particles.

B. AAV EF1α-Angiostatin was Shown to Decrease Tumor Metastasis in the B16F10-luc Metastasis Model.

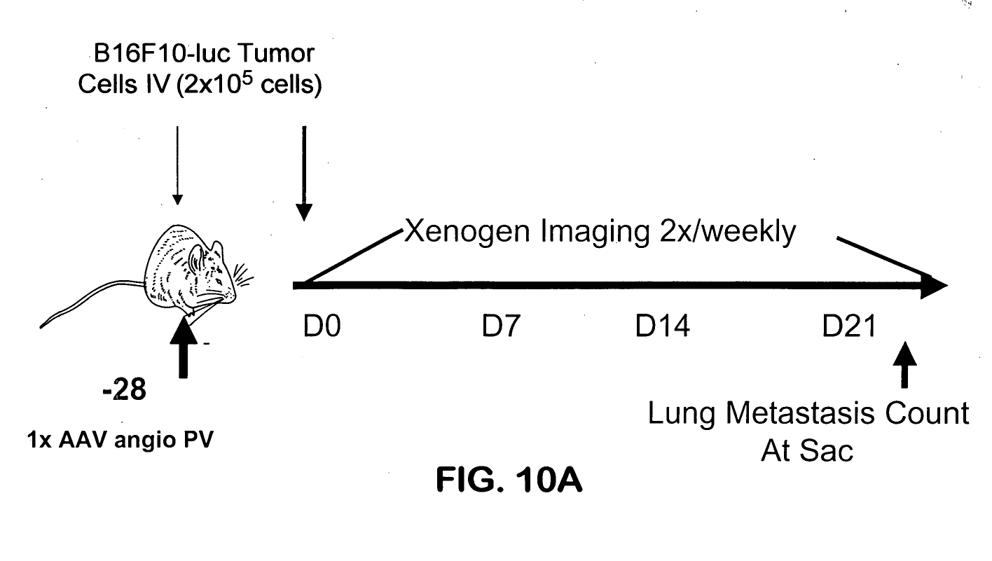

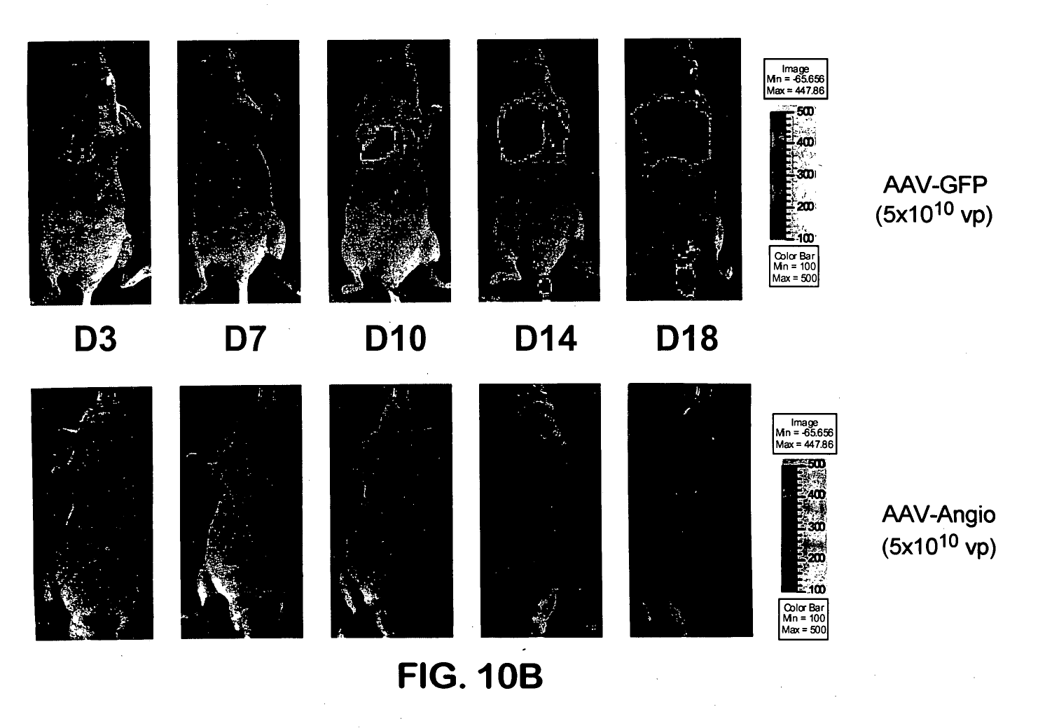

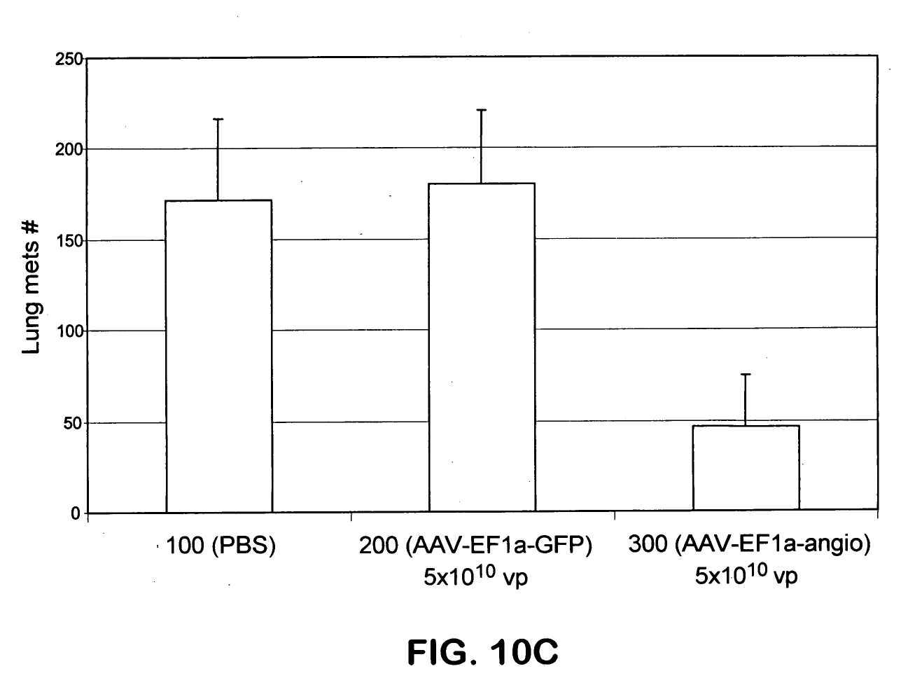

In one study, using the B16F10-luc metastasis model (shown in FIG. 10A), 5×10¹⁰ Vp of AAV- EF1-α-angisotatin or GFP were delivered via the portal vein into Balb/c nu/nu mice on day 1. Angiostatin expression was monitored weekly, and angiostatin levels were on the average 20-40 ng/ml in the AAV angiostatin group serum. Three weeks later, mice were injected with 5×10⁴ cells of B16F10-luc via the tail vein. Anti-tumor efficacy of AAV-angiostatin in the B16F10-luc metastasis model was evaluated by sacrificing all mice three weeks after tumor cell injection and harvesting the lungs for metastasis assessment. AAV-angiostatin treatment reduced surface lung metastasis nodules (n—6, p<0.005 in Student t-test) by 70%. Average Angiostatin expression in mice serum before sacrifice was about 20 ng/ml in the AAV-angiostatin injected mice. Similar animal experiments were repeated at least 3 times.

In a related study, using the B16F10-luc metastasis model, mice were monitored for 18 days following intravenous injection of 2×105 B16F10-luc cells (FIG. 10B). In vivo luminescence monitoring of tumor burden in the lung via total CCD photon counts demonstrated a strong linear correlation with the manual lung metastasis nodule count in the B16F10-luc metastasis model (FIG. 10C). This was demonstrated in nude mice as well as C57B1/6 mice (Sambucetti et al. and Chang et al., unpublished observations). On the average, AAV-angiostatin treated mice had significant less tumor burden evidenced by 2-3 fold reduction in luminescence signal as well (FIG. 10B).

C. AAV EF1α-Angiostatin was Shown to Increase Survival in the B16F10-luc Metastasis Model.

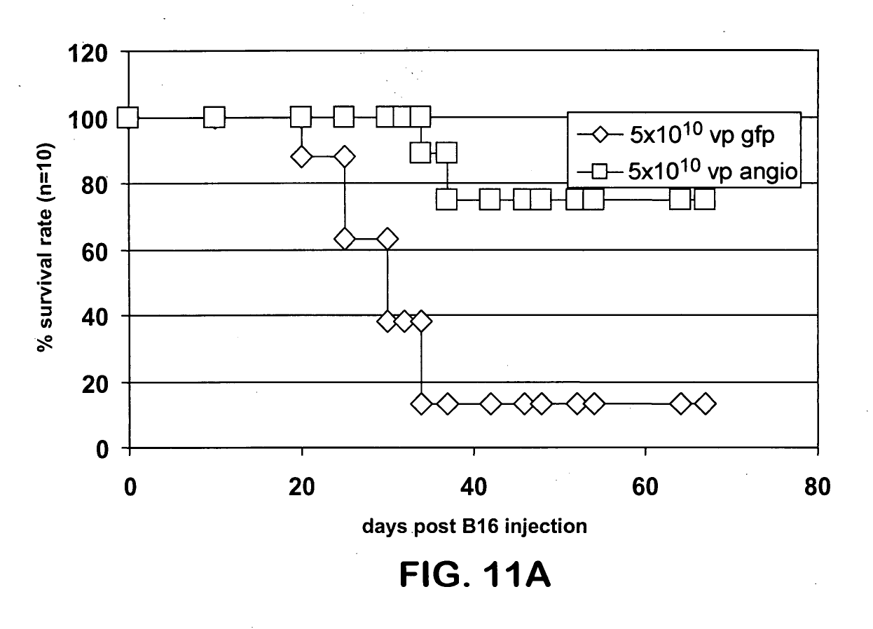

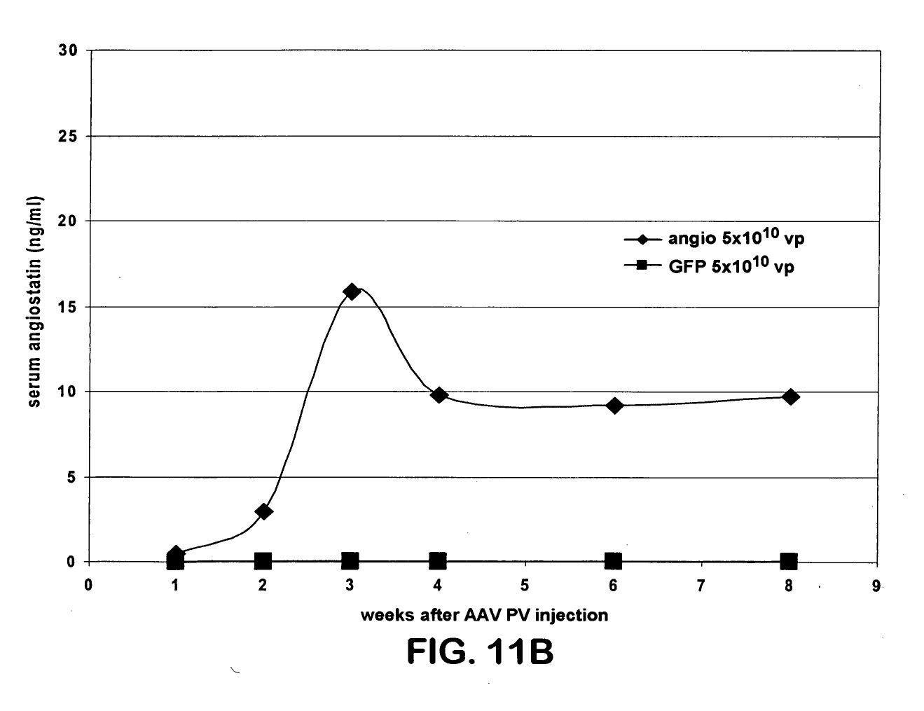

In a related study, directed to survival, 5×10¹⁰ rAAV vp encoding GFP or angiostatin were injected into Balb/c nu/nu mice on day 0 via the portal vein. Four weeks following gene transfer, 5×10⁴ B16F10-luc cells were injected intravenously into Balb/c nu/nu mice. Mice were monitored twice per week for in vivo bioluminescence with a Xenogen Imaging System to evaluate tumor burden and for health condition until they were sacrificed due to extensive tumor burden (FIG. 11A). Serum angiostatin levels from AAV EF1α-angiostatin and AAV EF1α-GFP transduced mice were monitored over time for 10 weeks. The results show that AAV EF1α-Angiostatin prolonged survival in the B16F10 metastasis model (FIG. 11A). At 50 days after B16 tumor cell injection about 70% of the angiostatin treated mice were still alive whereas less than 20% of the GFP treated mice survived (n=10). AAV-Angiostatin prolonged survival of mice significantly in this B16F10 metastasis survival study. The angiostatin expression from AAV-angiostatin, AAV-GFP delivered mice was monitored over time for 8 weeks (70 days) after AAV injection. AAV-angiostatin treated mice had a serum angiostatin concentration of 10-20 ng/ml (FIG. 11B).

In a dose response study of AAV EF1α-angiostatin in the B16F10 model, the results indicated that when 1×10¹⁰, 5×10¹⁰, 1×10¹¹ and 2×10¹⁰ AAV EF1α-angiostatin rAAV vp were injected into the mice, the best survival was observed with a dose of 5×10¹¹ Vp.

D. Increased Apoptosis Of Tumors Following AAV EF1 a-Angiostatin Gene Transfer.

Angiostatin protein delivery has been shown to induce tumor apoptosis in tumor models, as further described above. Tumor cell apoptosis was evaluated in mice by injection of 5×10¹⁰ vp of AAV- EF1-α-angisotatin or GFP via the portal vein of Balb/c nu/nu mice on day 1. Angiostatin expression was monitored weekly, and angiostatin levels were on the average 20-40 ng/ml. This steady state human angiostatin concentration was consistent with several other animal model studies. Three weeks later, mice were injected with 2×10⁵ B16B1/6 cells via the tail vein. Three weeks after the tumor cell injections, lungs were harvested, and flash frozen. Lungs with visible black lesions were sectioned, and stained for routine H&E for cell morphology. Tumor cell apoptosis was then quantified in situ with fluorescein-TUNEL staining (see Materials and Methods). These sections were simultaneously stained with DAPI for nuclear staining. The results indicated a marked increase of apoptosis within the tumor capsule in the AAV-angiostatin treated groups compared to the mock PBS injected groups or AAV control groups expressing GFP.

E. AAV EF1 α-Angiostatin Reduces Tumor Burden in the LLC-Resection Tumor Model.

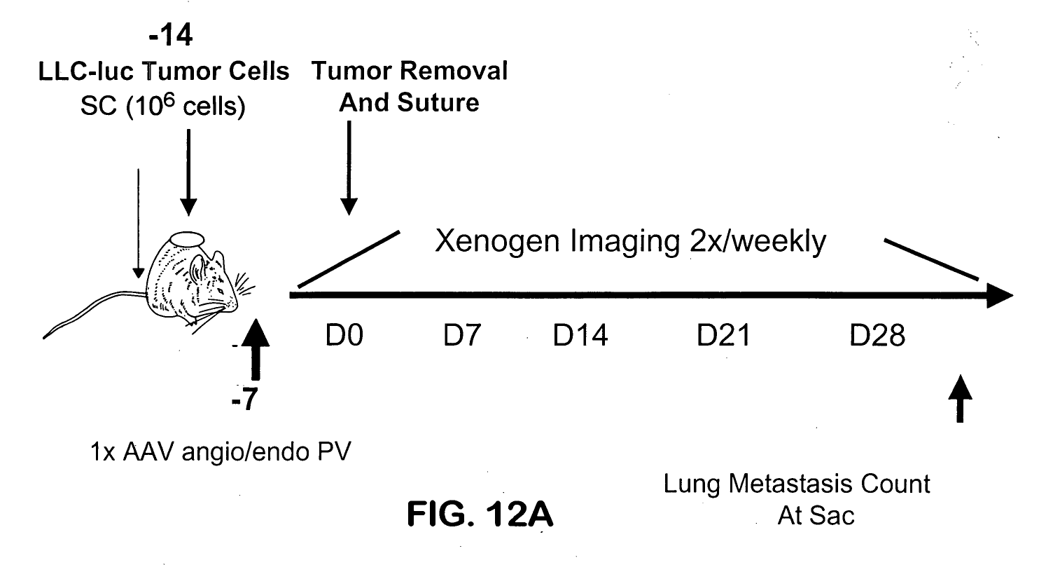

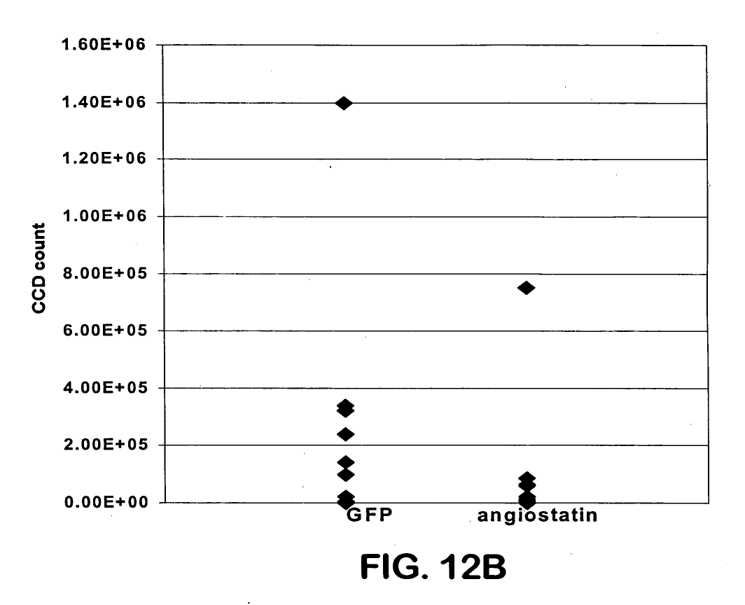

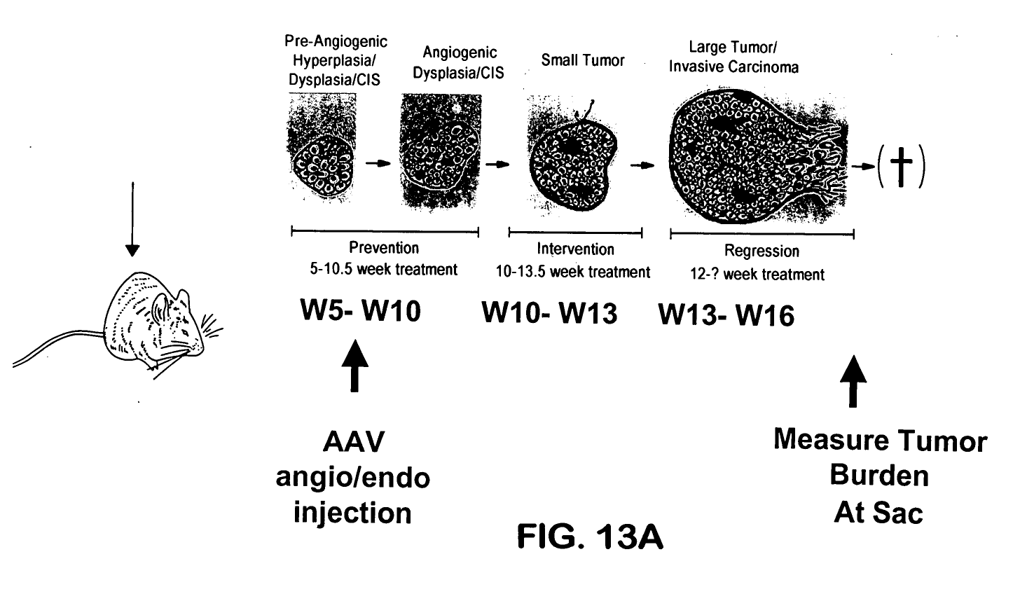

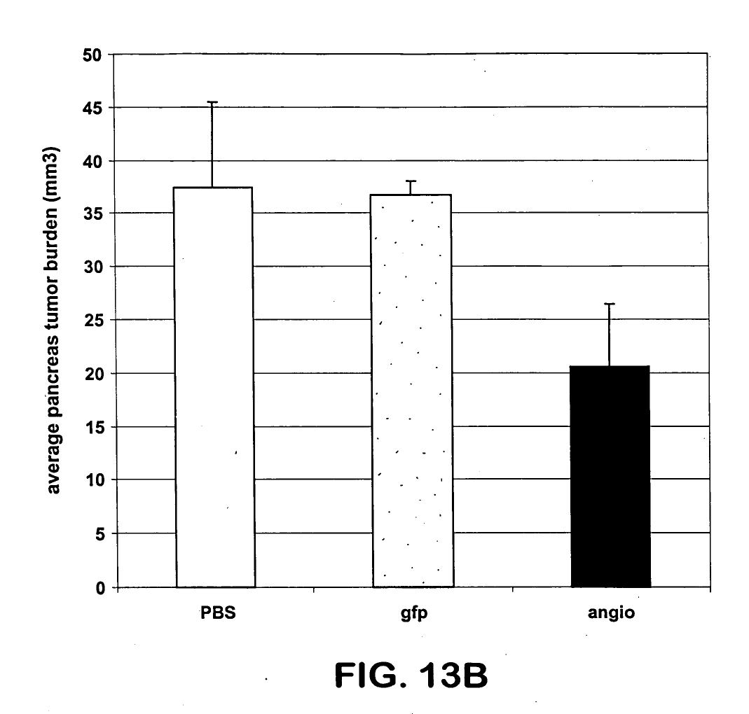

10⁶ Lewis lung carcinoma cells (10⁶) were injected subcutaneously on the back of Balb/c nu/nu mice (FIG. 12A). One week after implantation, all mice that had developed primary tumors were injected with 5×10¹⁰ vp of AAV-angiostatin or AAV-GFP. Two weeks later the primary tumors were resected and sutured to simulate a clinical situation. Lung metastasis appeared in a week. The appearance of metastasis was monitored in vivo with Xenogen Imaging (n=10) and CCD photon counts were evaluated 2 weeks after primary resection (FIG. 12B).

F. AAV EF1 α-Angiostatin Reduces Tumor Burden in the RIP-Tag Spontaneous Pancreatic Islet Carcinoma Model.

The RIP-Tag spontaneous pancreatic islet carcinoma model employs transgenic mice that have a rat insulin promoter (RIP) driven SV-40 antigen develop islet cell carcinomas as a result of the simian virus 40 oncogene expression in pancreatic islet cells. 1.5×10¹¹ AAV-angiostatin or AAV-GFP vectors viral particles were delivered via portal vein injection into 7-week-old RIP-Tag mice (FIG. 13A). Animals were sacrificed at week 13 and evaluated for pancreatic islet tumor burden. The results showed a lower tumor burden in mice injected with AAV-angiostatin (n=6)(FIG. 13B).

TABLE 3
Brief Table Of The Sequences.

| SEQ | |
|---|---|
| ID | |
| NO | Description |
| 1 | Human plasminogen 2732 bp mRNA sequence |
| | (found at GenBank Accession #: X05199) |
| 2 | Angiostatin K1-5 nucleic acid sequence: nucleotides 346-1800 of the human plasminogen coding |
| | sequence found at GenBank Accession #: X05199 |
| 3 | Angiostatin K1-3 nucleic acid sequence nucleotides 346-1128 of the human plasminogen coding |
| | sequence found at GenBank Accession #: X05199 |
| 4 | Plasminogen amino acid sequence (810 aa) (GenBank Accession #: P00747) |
| 5 | Angiostatin K1-3 protein sequence: amino acids 98-357 of the human plasminogen amino acid |
| | sequence |
| | VYLSECKTGNGKNYRGTMSKTKNGITCQKWSSTSPHRPRFSPATHPSEGLEENYCRNPDNDPQGPWCYTTDPEKRYDYCDILECEEECMHCS |
| | GENYDGKISKTMSGLECQAWDSQSPHAHGYIPSKFPNKNLKKNYCRNPDRELRPWCFTTDPNKRWELCDIPRCTTPPPSSGPTYQCLKGTGE |
| | NYRGNVAVTVSGHTCQHWSAQTPHTHNRTPENFPCKNLDENYCRNPDGKRAPWCHTTNSQVRWEYCKIPSCDSSPV |
| 6 | truncated version of human Angiostatin-consisting of amino acids 1-333 of human plasminogen |
| | 1 MDHKEVILLF LLLLKPGQGD SLDGYISTQG ASLFSLTKKQ LAAGGVSDCL AKCEGETDFV |
| | 61 CRSFQYHSKE QQCVIMAENS KTSSIIRMRD VILFEKRVYL SECKTGIGNG YRGTMSRTKS |
| | 121 GVACQKWGAT FPHVPNYSPS THPNEGLEEN YCRNPDNDEQ GPWCYTTDPD KRYDYCNIPE |
| | 181 CEEECMYCSG EKYEGKISKT MSGLDCQAWD SQSPHAHGYI PAKFPSKNLK MNYCHNPDGE |
| | 241 PRPWCFTTDP TKRWEYCDIP RCTTPPPPPS PTYQCLKGRG ENYRGTVSVT VSGKTCQRWS |
| | 301 EQTPHRHNRT PENFPCKNLE ENYCRNPDGE TAP |
| 7 | Angiostatin 1-5 protein sequence: amino acids 98-581 of the human plasminogen amino acid |
| | seqeucne found at GenBank Accession #: P00747 |
| 8 | Angio Forward |
| | 5′-CGTACCTAGGGAATTCAAGCTTACTAGTGCCGCCGCCATGGATGCAATGAAGAGAGG-3′ |
| 9 | Angio Reverse |
| | 5′ GTCAGTCGACTCTAGAAGTGGATCTTTAAGGTGGTGCTG 3′ |
| 10 | Human endostatin coding sequence (nucleotides 3509 to 4057 of GenBank |
| 11 | Human endostatin protein sequence 183 aa |
| | HSHRDFQPVLHLVALNSPLSGGMRGIRGADFQCFQQARAVGLAGTFRAFLSSRLQDLYSIVRRADRAAVPIVNLKDELLFPSWEALFSGSEG |
| | PLKPGARIFSFDGKDVLRHPTWPQKSVWHGSDPNGRRLTESYCETWRTEAPSATGQASSLLGGRLLGQSAASCHHAYIVLCIENSFMTASK |
| 12 | Angiostatin K1-3 signal sequence (nucleic acid) |
| | ATGGATGCAATGAAGAGAGGGCTCTGCTGTGTGCTGCTGCTGTGTGGAGCAGTCTTCGTTTCGCCCAGCGGTACCGGATCCTTATTTGAAAA |
| | GAAA |
| 13 | Angiostatin K1-3 signal sequence (amino acid) |
| | MDAMKRGLCCVLLLCGAVFVSPSGTGSLFEKK |

It is claimed:

1. A recombinant viral vector for obtaining angiostatin activity, comprising:
a promoter capable of expressing human angiostatin operably linked to a structural gene encoding one or more domains of human angiostatin.

2. The vector of claim 1, wherein said viral vector is a recombinant adenovirus (rAV) vector.

3. The vector of claim 2, wherein said viral vector is an E1/E4 deleted recombinant adenovirus (rAV) vector.

4. The vector of claim 1, wherein said viral vector is an adeno-associated virus (rAAV) vector.

5. The vector of claim 1, wherein said promoter is the EF-1 alpha promoter.

6. A recombinant adenovirus (rAV) vector for expressing an anti-angiogenic compound, comprising:
a promoter capable of expressing said anti-angiogenic compound operably linked to a structural gene encoding a biologically active form of said anti-angiogenic compound.

7. The vector of claim 6, wherein said adenovirus vector is an E1/E4 deleted recombinant adenovirus (rAV) vector.

8. The vector of claim 5, wherein said anti-angiogenic compound is angiostatin.

9. The vector of claim 5, wherein said anti-angiogenic compound is endostatin

10. The vector of claim 5, wherein said promoter is the EF-1 alpha promoter.

11. A recombinant adeno-associated virus (rAAV) for expressing an anti-angiogenic compound, comprising:
a promoter capable of expressing said anti-angiogenic compound operably linked to a structural gene encoding a biologically active form of said anti-angiogenic compound; and
two AAV inverted terminal repeats,
wherein said inverted terminal repeats flank the promoter and structural gene.

12. The vector of claim 9, wherein said anti-angiogenic compound is angiostatin.

13. The vector of claim 9, wherein said anti-angiogenic compound is endostatin

14. The vector of claim 9, wherein said promoter is the EF-1 alpha promoter.

15. A method of inhibiting angiogenesis in a mammalian subject, comprising:
administering a replication-defective viral vector comprising a DNA sequence encoding angiostatin operably linked to a promoter, wherein a biologically active form of angiostatin is expressed.

16. The method of claim 13, wherein said replication-defective viral vector is an adeno-associated viral (AAV) vector.

17. The method of claim 13, wherein said replication-defective viral vector is an adenoviral (AV) vector.

18. The method of claim 13, wherein said promoter is the EF-1 alpha promoter.

19. The method of claim 13, wherein said replication-defective viral vector is administered in vivo into the portal vasculature of said mammal.

20. The method of claim 13, wherein said replication-defective viral vector is administered intravenously to said mammal.

21. A method of inhibiting angiogenesis in a mammalian subject, comprising:
administering a recombinant adeno-associated virus (rAAV) vector comprising a DNA sequence encoding an anti-angiogenic compound operably linked to a promoter, wherein a biologically active form of said anti-angiogenic compound is expressed.

22. The method of claim 21, wherein said anti-angiogenic compound is angiostatin.

23. The method of claim 21, wherein said anti-angiogenic compound is endostatin.

24. The method of claim 21, wherein said promoter is the EF-1 alpha promoter.

25. The method of claim 21, wherein said administering comprises delivering said rAAV vector into the portal vasculature of said mammal.

26. The method of claim 21, wherein said administering comprises delivering said rAAV vector intravenously to said mammal.

27. A method of inhibiting angiogenesis in a mammalian subject, comprising:
administering a recombinant adenovirus (rAV) vector comprising a DNA sequence encoding an anti-angiogenic compound operably linked to a promoter to cells capable of expressing said compound, wherein a biologically active form of said anti-angiogenic compound is expressed.

28. The method of claim 26, wherein said anti-angiogenic compound is angiostatin.

29. The method of claim 26, wherein said anti-angiogenic compound is endostatin.

30. The method of claim 26, wherein said promoter is the EF-1 alpha promoter.

31. The method of claim 25, wherein said administering comprises delivering said rAAV vector into the portal vasculature of said mammal.

32. The method of claim 25, wherein said administering comprises delivering said rAAV vector intravenously to said mammal.
